(19) United States
(12) Patent Application Publication
(10) Pub. No.: US 2005/0250709 A1
(43) Pub. Date: Nov. 10, 2005

(54) Anti-neoplastic agents, combination therapies and related methods

(75) Inventor: Mehran Khodadoust, Brookline, MA (US)

Correspondence Address: FISH & NEAVE IP GROUP;ROPES & GRAY LLP, ONE INTERNATIONAL PLACE, BOSTON, MA, 02110-2624, US

(73) Assignee: Bionaut Pharmaceuticals, Cambridge, MA (US)

(21) Appl. No.: 11/018,399

(22) Filed: Dec. 20, 2004

Related U.S. Application Data
(60) Provisional application No. 60/531,125, filed on Dec. 19, 2003.
(60) Provisional application No. 60/551,563, filed on Mar. 8, 2004.

(51) Int. Cl. A61K 31/704; A61K 31/155

(52) U.S. Cl. 514/26; 514/635

(57) ABSTRACT

In certain aspects, the invention relates to the discovery of novel compositions for use in the treatment of a neoplastic disorder. Further aspects of the invention relate to the discovery that cell signaling may be exploited to identify significant genes in cancer cell lines and to generate reporter gene systems that may be used, for example, to identify anti-neoplastic agents and effective combinations thereof.

### RELATED APPLICATIONS

This application claims the benefit of U.S. Provisional Application Ser. No. 60/531,125, filed Dec. 18, 2003, and claims the benefit of U.S. Provisional Application Ser. No. 60/551,563, filed Mar. 8, 2004. All the teachings of the above-referenced applications are incorporated herein by reference.

### BACKGROUND

Certain aspects of the invention relate to the field of anti-neoplastic agents. Certain aspects of the invention relate to the field of drug discovery and cell-based assay systems.

Traditionally, chemotherapeutic agents have been selected or designed to have increased activity against proliferating cells versus quiescent cells. For example, cisplatin agents tend to cause lethal alterations in the genomic material of dividing cells. Taxol and a variety of other chemotherapeutic agents target the cytoskeleton, and are generally thought to interfere with mitosis. While focusing on the hyperproliferative nature of cancer cells has been a successful approach to the design of anti-neoplastic agents, it is now possible to design agents that target specific molecular pathways.

It is increasingly common to target secreted factors that participate in cancer progression, such as growth factors and pro-angiogenic factors. The effectiveness of these pathway targeted drugs is becoming clear, with many now on the market or in clinical trials.

One limitation on the power of molecular targeting is the availability of selective assays and the limited number of known, relevant targets. The invention provides, among other things, novel cell-based assay systems and anti-neoplastic agents identified with such assay systems.

### SUMMARY

In certain aspects, the invention relates to the discovery of novel compositions for use in the treatment of a neoplastic disorder. Compositions formulated or packaged for use in treating a neoplastic disorder may include, for example, a member of the alexidine class of compounds or a member of the niclosamide class of compounds. In certain aspects, the disclosure provides combination anti-neoplastic therapies that employ two or more agents, as well as combinations formulated or packaged for use in treating a neoplastic disorder. Combination anti-neoplastic therapies may include two or more agents selected from among the following categories: a redox effector, a cardiac glycoside, a steroid signal modulator and an growth factor antagonist. For example, a combination therapy may include a compound that is a redox effector and a compound that is a steroid signal modulator. As another example, a combination therapy may include a compound that is a cardiac glycoside and a compound that is a steroid signal modulator. Further aspects of the invention relate to novel formulations of certain neoplastic agents disclosed herein, such as formulations designed to increase serum half-life or formulations for a particular mode of delivery (e.g., topical or systemic). Further aspects of the invention relate to the discovery that cell signaling may be exploited to identify significant genes in cancer cell lines and to generate reporter gene systems that may be used, for example, to identify anti-neoplastic agents and effective combinations thereof. As demonstrated herein, there is a surprisingly high correlation between the tendency of an agent to decrease the expression of a HIF-responsive reporter gene in a cancer cell line and the tendency of the agent to have an antiproliferative effect. Accordingly, reporter gene cell lines herein may be employed in screening assays to identify test agents having antineoplastic effects.

In certain embodiments, the disclosure provides methods for treating a neoplastic disorder by administering to a patient a member of the alexidine class of compounds. A compound of the alexidine class of compounds may be formulated in a pharmaceutical composition for use in treating a neoplastic disorder, and such a composition may comprise one or more pharmaceutically acceptable excipients. In certain embodiments, the disclosure provides a packaged pharmaceutical for use in treating a neoplastic disorder, comprising: a pharmaceutical composition comprising a member of the alexidine class and a pharmaceutically acceptable carrier; and instructions for use. A pharmaceutical composition may comprise an additional anti-neoplastic agent, such as a cardiac glycoside, a redox effector, a steroid signal modulator or a growth factor antagonist. In preferred embodiments, the neoplastic disorder to be treated comprises, or is suspected of comprising, EGF-responsive tumor cells, particularly EGFR-positive tumor cells. In certain embodiments, the neoplastic disorder to be treated comprises or is suspected of comprising HIF-positive cells (e.g., cells positive for a HIF-1α or HIF-2 polypeptide). Optionally, the neoplastic disorder to be treated is selected from among: pancreatic cancer, lung cancer (e.g., non-small cell lung carcinoma), colon cancer, prostate cancer, cervical cancer, renal cancer, uterine cancer and breast cancer.

In certain embodiments, the disclosure provides methods for treating a neoplastic disorder by administering a cardiac glycoside. In a further embodiment, cardiac glycoside may be formulated in a pharmaceutical composition for use in treating a neoplastic disorder, and such a composition may comprise one or more pharmaceutical excipients. In certain embodiments, the disclosure provides a packaged pharmaceutical for the treatment of a neoplastic disorder, comprising: a pharmaceutical composition comprising ouabain and a pharmaceutically acceptable carrier, and instructions for use. The pharmaceutical composition may optionally comprise an additional anti-neoplastic agent, such as a redox effector, a steroid signal modulator or a growth factor antagonist. Preferred additional anti-neoplastic agents include niclosamide and bicalutamide (Casodex®). In certain embodiments, the neoplastic disorder to be treated comprises, or is suspected of comprising, EGF-responsive tumor cells, particularly EGFR-positive tumor cells. In certain embodiments, the neoplastic disorder to be treated comprises or is suspected of comprising HIF-positive cells (e.g., cells positive for a HIF-1 or HIF-2 polypeptide). Optionally, the neoplastic disorder to be treated is selected from among: pancreatic cancer, lung cancer (e.g., non-small cell lung carcinoma), colon cancer, prostate cancer, cervical cancer, renal cancer, uterine cancer and breast cancer.

In certain embodiments, the disclosure provides methods for treating a neoplastic disorder by administering to a patient a member of the niclosamide class of compounds. A compound of the niclosamide class of compounds may be formulated in a pharmaceutical composition for use in treating a neoplastic disorder, and such a composition may comprise one or more pharmaceutically acceptable excipients. In certain embodiments, the disclosure provides a packaged pharmaceutical for use in treating a neoplastic disorder, comprising: a pharmaceutical composition comprising a member of the niclosamide class and a pharmaceutically acceptable carrier; and instructions for use. A pharmaceutical composition may comprise an additional anti-neoplastic agent, such as a a cardiac glycoside, a redox effector, a steroid signal modulator or a growth factor antagonist. In preferred embodiments, the neoplastic disorder to be treated comprises, or is suspected of comprising, EGF-responsive tumor cells, particularly EGFR-positive tumor cells. In certain embodiments, the neoplastic disorder to be treated comprises or is suspected of comprising HIF-positive cells (e.g., cells positive for a HIF-1 or HIF-2 polypeptide). Optionally, the neoplastic disorder to be treated is selected from among: pancreatic cancer, lung cancer (e.g., non-small cell lung carcinoma), colon cancer, prostate cancer, cervical cancer, renal cancer, uterine cancer and breast cancer.

In certain embodiments, the disclosure provides methods for treating a neoplastic disorder by administering a combination regimen including two agents selected from among the following categories: a cardiac glycoside, a redox effector; a steroid signal modulator and a growth factor antagonist. A combination regimen will generally involve administration of the agents in such a manner as to achieve an effect that is more beneficial than the administration of either compound alone. This may involve temporally concurrent, overlapping or separate administration of the agents, so long as there is some overlapping effect. Optionally, the combined agents may be formulated into a convenient, combined pharmaceutical formulation comprising a pharmaceutically effective carrier and two agents selected from among the following categories: a redox effector; a steroid signal modulator; and a growth factor antagonist. Optionally, the redox effector, steroid signal modulator and/or cardiac glycoside is an agent that decreases HIF-1 or HIF-2 activity, either by affecting transcription, translation, steady state accumulation, degradation or other post translational effect on HIF-1, HIF-2 or a constituent protein thereof. Optionally a redox effector is: an alexidine class member; a clofazimine class member, an electron transport inhibitor, a cytochrome P450 inhibitor and an electron acceptor. Optionally, the steroid signal modulator is selected from among: a steroid hormone and a steroidogenesis inhibitor. A pharmaceutical formulation may be packaged with instructions for use in treating a neoplastic disorder. While combination therapies disclosed herein are expected to have broad applicability in neoplastic disorders, such a therapy may be preferably administered to a patient suffering from a neoplastic disorder that includes, or is suspected of including, an EGF responsive tumor cell, such as an EGFR-positive tumor cell. In certain embodiments, the neoplastic disorder to be treated comprises or is suspected of comprising HIF-positive cells (e.g., cells positive for a HIF-1 or HIF-2 polypeptide).

In yet a further aspect, the invention relates to the discovery of cells comprising a reporter gene and/or one or more selectable markers that are expressed in response to extracellular signaling. In certain embodiments, the disclosure provides cells comprising: a nucleic acid construct which includes one or more of the following: a positive selection marker, a negative selection marker and a reporter gene, wherein expression of the reporter and or selection markers are regulated by extracellular signaling. In a preferred embodiment, the construct comprises both a positive selection marker and a negative selection marker, and optionally a reporter gene. Whether a selectable marker or reporter gene is responsive to extracellular signaling may be characterized in a variety of ways, including, for example, a comparison of expression in a high cell density cell culture and in a low cell density cell culture, where a responsive gene will be expressed at a higher level in the high cell density cell culture. Optionally, expression of the marker(s) and/or reporter gene are regulated by a hypoxia-responsive transcription factor, such as HIF-1 or HIF-2. Optionally, expression of the marker(s) and/or reporter gene are inhibited by an EGF antagonist or a compound such as a redox effector or steroid signaling modulator.

In certain embodiments, the disclosure provides methods for identifying candidate anti-neoplastic agents by evaluating the effect of the candidate agent on the expression of a gene, preferably a reporter gene, that is responsive to extracellular signaling. A method may comprise: (a) culturing a cell comprising an extracellular signaling responsive reporter gene under conditions wherein the reporter gene is detectably expressed; (b) contacting the cell with a test agent; (c) detecting expression of the reporter gene; and (d) comparing the expression of the reporter gene in (c) to an untreated reference, wherein a test agent that causes a decrease in the expression of the reporter gene relative to the untreated reference is a candidate anti-neoplastic agent. A reference may be, for example, a simultaneous untreated control cell or any record of such previously measured control cell. Optionally, the test agent may be selected from among the following: an EGF antagonist, a cardiac glycoside, a redox effector and a steroid signaling modulator. In a preferred embodiment, the cell is derived from an established cancer cell line, such as an established cell line of one of the following cancer types: pancreatic cancer, lung cancer (e.g., non-small cell lung carcinoma), colon cancer, prostate cancer, cervical cancer, renal cancer, uterine cancer and breast cancer.

In certain embodiments, the disclosure provides methods for identifying a candidate anti-neoplastic agent, the method comprising: (a) contacting a cell with a test agent, wherein the cell comprises a reporter gene that is responsive to extracellular signaling; (b) detecting expression of the reporter gene; and (c) comparing the expression of the reporter gene in (b) to an untreated reference, wherein a test agent that causes a decrease in the expression of the reporter gene relative to the untreated reference is a candidate anti-neoplastic agent.

In further embodiments, the disclosure provides methods for identifying effective anti-neoplastic combinations, the method comprising: (a) culturing a cell comprising an extracellular signaling responsive reporter gene under conditions wherein the reporter gene is detectably expressed; (b) contacting the cell with a first candidate anti-neoplastic agent and a second candidate anti-neoplastic agent; (c) detecting expression of the reporter gene; and (d) comparing the expression of the reporter gene in (c) to an appropriate reference, wherein, a combination of first candidate anti-neoplastic agent and second candidate anti-neoplastic agent that decreases expression of the reporter gene more than either anti-neoplastic agent alone is an effective anti-neoplastic combination.

In certain embodiments, the disclosure provides methods of generating cancer cells expressing a reporter gene that is responsive to extracellular cell signaling, the method comprising: (a) transfecting a cancer cell line with a nucleic acid construct including a positive selection marker, a negative selection marker and a reporter gene, wherein expression of the positive selection marker, the negative selection marker and the reporter gene is coordinately regulated; (b) culturing the transformants to a high cell density; (c) selecting for cells expressing the negative selection marker, thereby eliminating a substantial portion of the cultured cells; and (d) selecting for cells that do not express the positive selection marker, wherein cells selected in (d) are cancer cells that express a reporter gene in response to extracellular cell signaling.

In certain aspects, the invention provides methods for operating a pharmaceuticals business. Such a method may comprise employing an assay system disclosed herein to identify an effective anti-neoplastic agent or an effective combination. The identified agent or combination may be tested for efficacy and/or safety in an animal model and in humans. Such data may also be pre-existing for those agents that have been previously used in humans. The identified agent or composition may be packaged for sale and/or licensed to another party for production and distribution. In certain aspects, the invention provides a medical care reimbursement business, wherein a claim is received relating to the use of an agent or composition disclosed herein for the treatment of a neoplastic disorder; the claim is evaluated and as appropriate, paid in full, in part or denied.

### BRIEF DESCRIPTION OF THE DRAWINGS

FIG. 1 is a schematic of a nucleic acid construct for use in creating reporter cell lines that are responsive to, for example, extracellular signaling. Generally, such a construct may comprise a negative selection marker (here, thymidylate kinase “TK”), a positive selection marker (here, zeocin resistance “Zeo”) and a reporter gene (here, beta-galactosidase “β-gal”).

FIG. 2 is a schematic of a method for identifying genomic inserts that are responsive to an agent, such as a growth factor. The method as shown will also identify genomic inserts that are responsive to extracellular signaling, and such inserts may also be identified by omitting the addition of agent in the first step of the depicted method.

FIG. 3 shows basal levels of reporter gene expression in A549 clones carrying an insert that is responsive to IGF-1, EGF and insulin (A7N1C1) and extracellular signaling (A7E1A5 and A6E2A4). A549 is a well-established human non-small cell lung carcinoma cell line.

FIG. 4 shows the responsiveness of the A7N1C1 cell line to various growth factor antagonists. Iressa and AG1478 are EGF antagonists. AG1024 is an insulin antagonist.

FIG. 5 shows the responsiveness of the A7N1C1 cell line to combinations of growth factor antagonists.

FIG. 6 shows the effects of an EGF antagonist on EGF, insulin and IGF responsive gene expression in the A7N1C1 cell line.

FIG. 7 shows the effects of an EGF antagonist on extracellular signaling responsive cell lines.

FIG. 8 shows the effects of an EGF antagonist and two tyrphostins on extracellular signaling-responsive gene expression.

FIG. 9 shows the effects of an EGF antagonist on EGF, insulin and IGF responsive gene expression in the A7E1A5 extracellular signaling-responsive cell line.

FIG. 10 shows a schematic for identifying the location of genomic inserts of interest, by which factor or extracellular-signaling responsive genes may be identified.

FIG. 11 illustrates the use of an EGF responsive cell line to screen for compounds affecting EGF regulate gene expression.

FIG. 12 illustrates the identification of agents having potency roughly equal to or greater than that of the EGF inhibitor, Iressa.

FIG. 13 shows the effects of the inhibition of HIF-1 and HIF-2 on expression of a reporter gene in cancer cell lines, demonstrating the successful development of HIF-responsive cancer cell lines.

FIG. 14 illustrates the use of the HIF-responsive reporter cell line in the identification of candidate lung cancer treatments.

FIG. 15 shows the additive inhibitory effect of ouabain (a cardiac glycoside) and niclosamide (a redox effector) as demonstrated by reduction of reporter activity. FIG. 15 also shows that ouabain and niclosamide act through different mechanistic pathways, with the redox effector causing a decrease in the level of HIF-2 protein.

FIG. 16 shows the antiproliferative effect of ouabain on a variety of cancer cell types.

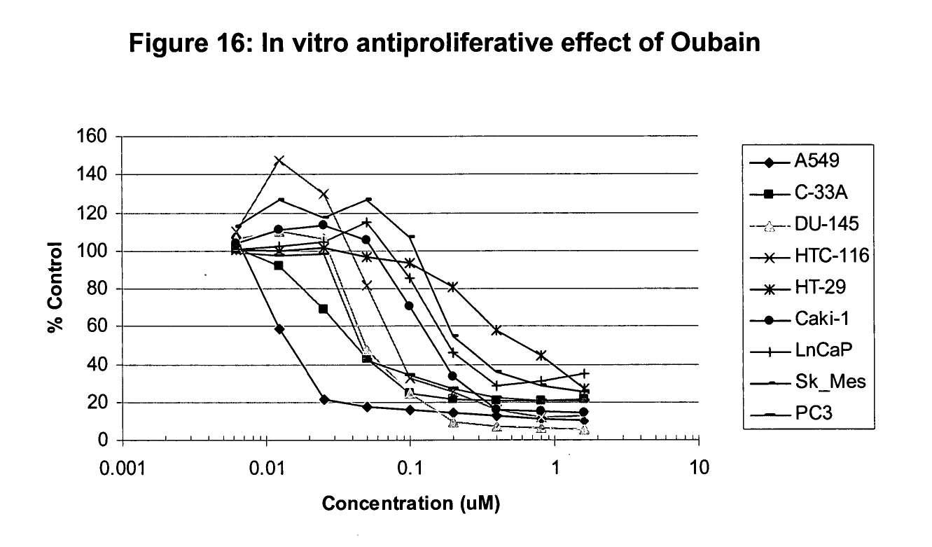

FIG. 17 shows the antiproliferative effect of niclosamide on a variety of cancer cell types.

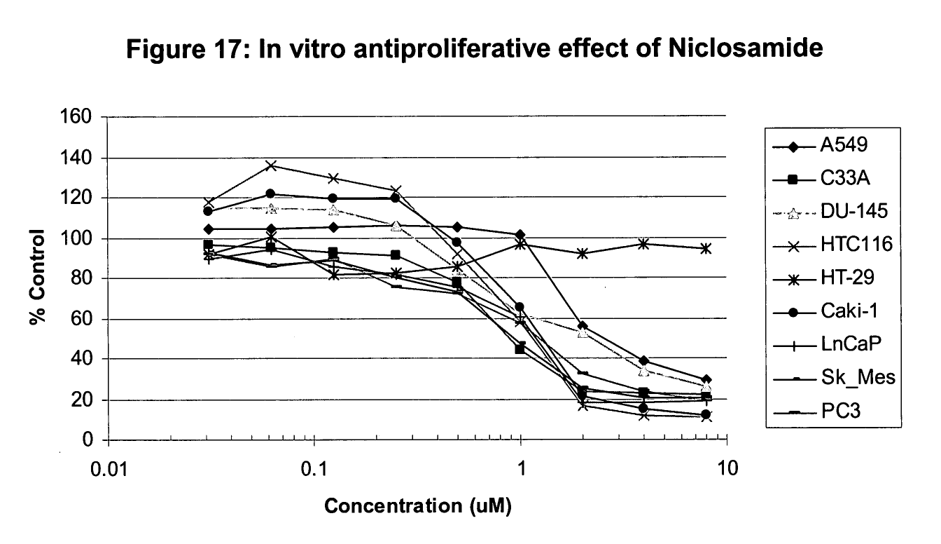

FIG. 18 shows the combined inhibitory effect of niclosamide and casodex as demonstrated by reduction of reporter activity.

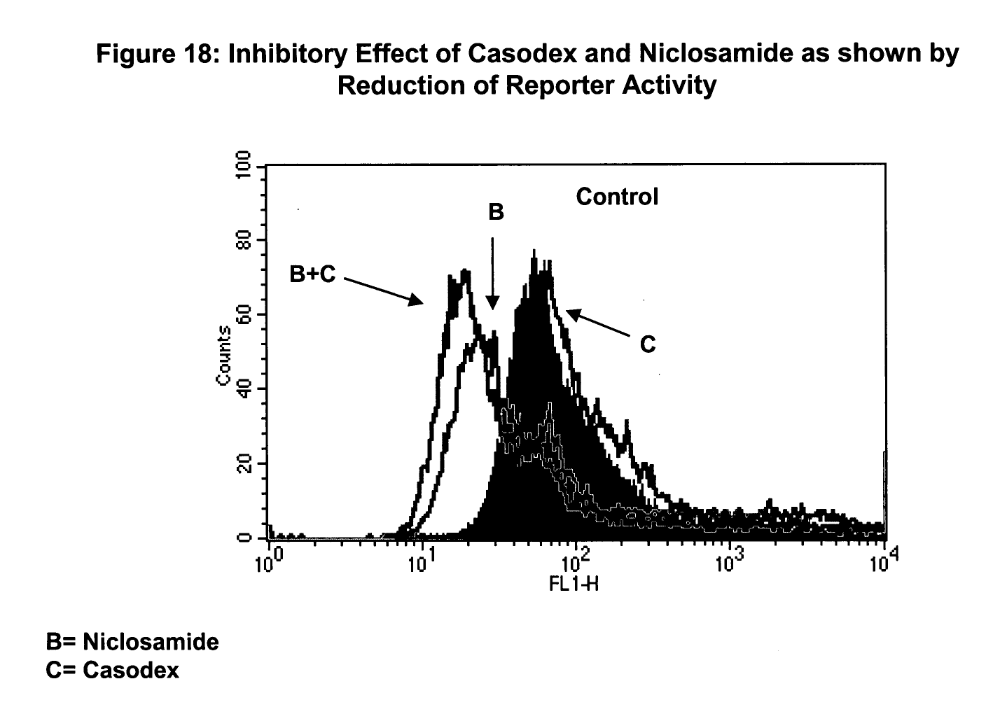

FIG. 19 shows the combined inhibitory effect of ouabain and casodex as demonstrated by reduction of reporter activity.

FIG. 20 shows the combined antiproliferative effect of ouabain and casodex.

### DETAILED DESCRIPTION

1. Overview

In part, the invention is related to the discovery of cancer cell lines carrying reporter genes that are responsive to one or more global hypoxia-responsive regulators, such as the HIF-1 and HIF-2 multimeric transcription factors (“HIF responsive”). A subset of the disclosed cell lines are responsive to general extracellular signaling (e.g., paracrine or autocrine signaling) and a subset of the disclosed cell lines are responsive to one or particular growth factors, such as EGF, IGF and insulin. As demonstrated herein, cells that are responsive to extracellular signaling and/or hypoxia regulators are useful in identifying anti-neoplastic agents, and combinations thereof. Accordingly, aspects of the invention provide methods for making such cell lines and the use of such cell lines in drug discovery and characterization. Strikingly, Applicants have demonstrated that candidate agents that prove effective in HIF-responsive reporter gene assays are likely to also have antiproliferative effects. Accordingly, the HIF-responsive cell lines disclosed herein are highly predictive.

In part, the invention is related to the discovery of anti-neoplastic agents and combinations and formulations of such agents. Applicants have discovered, from among thousands of tested compounds, three categories of compounds that have potency against cancer cell lines that are hypoxia and/or extracellular signaling responsive. These categories of compounds are redox effectors, cardiac glycosides, and steroid signal modulators. While these compounds, used alone, show a potency on par with that of approved chemotherapeutic agents, the invention further relates to the discovery that members of these three categories of compounds have effects that are additive or cumulative and in some instances, cooperative.

While not wishing to be bound to any particular mechanism, the results described here indicate that a wide range of cancers may be treated with a combination therapy approach that targets (1) aspects of transcriptional regulation, particularly as mediated by sterol signaling and steroid hormone receptors (whether nuclear or membrane bound), and (2) aspects of cellular redox state. It is expected that redox effectors act, directly or indirectly, to decrease the level and/or activity of a global hypoxia regulator. For example, HIF-1 is composed of two subunits, HIF-1α and HIF-1β. The HIF-1α subunit is controlled in a variety of ways, and notably at the transcriptional level by one or more growth factors and at the level of protein stability by von-Hippel Lindau factor (VHL) and ubiquitin-mediated protein degradation. Exposure of cells to a variety of redox reactive agents rapidly inhibits HIF-1α degradation, permitting rapid accumulation of the protein and subsequent activation of hypoxia responsive genes. HIF-1 and HIF-2 are global transcriptional regulators and are commonly activated in cancers. In fact, HIF-1 is a marker for poor prognosis in a variety of cancers, including colorectal cancer and cervical cancer. HIF-1 stimulates a general angiogenic pathway, including VEGF induction, as well as a cytoskeletal remodeling that is associated with cancer cell motility and invasiveness. Accordingly, a variety of the anti-neoplastic agents disclosed herein may affect cancer cells by downregulating HIF-1α or a similar hypoxia regulator. Hypoxia is a common condition in a wide range of tumors, and particularly solid tumors, where expansion of the vasculature is insufficient to keep up with the growth of the tumor. Moreover, a variety of cancers accumulate mutations that produce a constitutive “hypoxia-like” phenotype, even in the presence of normal oxygen tension (“normoxia”), although such cancers also tend to develop additional mutations, such as p53 mutations, that eliminate any of the pro-apoptotic or anti-proliferative reactions that would normally be triggered by hypoxia regulators.

Consistent with this model, Applicants have demonstrated that niclosamide, a representative of the class of redox effectors, and ouabain, a representative of the cardiac glycosides, have cumulative antiproliferative effects on cancer cell lines when used in combination. Furthermore, the redox effector causes a decrease in the level of HIF-2α protein while the cardiac glycoside does not, indicating that these two categories of compounds achieve an effect on HIF-responsive gene expression through different mechanisms.

In preferred embodiments, the agents and combinations described herein are used in treating: pancreatic cancer, lung cancer (e.g., non-small cell lung carcinoma), colon cancer, prostate cancer, cervical cancer, renal cancer, uterine cancer or breast cancer. However, it is expected that such combinations will have wide applicability in many other cancers, including, as a partial list, head and neck cancers (e.g., head and neck squamous cell carcinoma), cervical cancers, Wilms' tumor, liver cancers (e.g., hepatomas), ovarian cancers (e.g., ovarian carcinoma), cancers of the central nervous system and melanoma. In addition to cancers, the anti-neoplastic agents, may, where appropriate be used to treat other disorders characterized by unwanted cellular proliferation, particularly in treatment of pre-cancerous growths.

Combination therapies may be administered in any manner that takes advantage of the benefits of combination. Typically, the combined agents will be pre-mixed as a single formulation, however, the combined agents may also be administered separately. Where administered separately, the agents may be administered simultaneously or at different times, so long as some benefit of combination is retained.

In certain embodiments, the invention provides packaged pharmaceuticals. Such packaged pharmaceuticals will generally comprise a pharmaceutical composition and instruction for use. The phrase “instruction for use” is intended to encompass any communicative material (e.g., writing, visual aid, software, etc.) that conveys an intent to use the packaged pharmaceutical for treating a patient having, or suspected of having, a neoplasia. “Instructions for use” will commonly be a label, indicating the appropriate clinical indication, and may optionally include dosage information and/or safety information.

Unless defined otherwise, all technical and scientific terms used herein have the same meaning as commonly understood by one of ordinary skill in the art to which this invention belongs. The articles “a” and “an” are used herein to refer to one or to more than one (i.e., to at least one) of the grammatical object of the article, unless context clearly indicates otherwise. By way of example, “an element” means one element or more than one element. The term “including” is used herein to mean, and is used interchangeably with, the phrase “including but not limited to”. The term “or” is used herein to mean, and is used interchangeably with, the term “and/or”, unless context clearly indicates otherwise.

2. Therapeutic Agents

In certain aspects, the invention provides novel uses and formulations for certain therapeutic agents in the treatment of neoplastic disorders, such as cancer. In certain embodiments, the invention provides novel combination therapies.

As disclosed herein, alexidine is effective in suppressing cancer related gene expression in various cancer cell lines. For example, alexidine is effective in suppressing EGF, insulin and/or IGF-responsive gene expression in various growth factor responsive cancer cell lines. As another example, alexidine is effective in suppressing HIF mediated gene expression in certain cancer cell lines. Accordingly, alexidines and related compounds may be used in treatments for cancer, alone or in combination with other agents. In preferred embodiments, the cancer to be treated will be HIF-positive.

Alexidine and related compounds (the “alexidine class”) are generally bis-biguanides, amidinoureas and amidinothioureas. Compounds of the alexidine class may be selected on the basis of decreased expression of one or more EGF-responsive genes in a cancer cell line. Compounds of the alexidine class may also be selected on the basis of decreased expression of one or more HIF-responsive genes in a cancer cell line. Examples of alexidines include those hexamethylene-bis-biguanides disclosed in U.S. Pat. No. 4,022,834, and French Patent No. FR 1463818, and amidinoureas and amidinothioureas disclosed in U.S. Pat. No. 4,022,962. Examples of alexidine structures include: 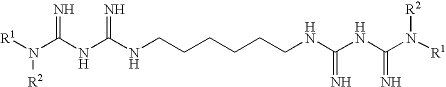
where R¹ represents H, and
R² represents hexyl or pentyl groups substituted with one or two methyl groups, or represents a cycloalkyl group having more than six carbon atoms, a lower alkyl-cycloalkyl group or a cycloalkyl-lower alkyl group, or 4-chlorophenyl (chlorheximine),
or R¹ and R² together with the adjacent N-atom represents a 3-azabicyclo-[3,2,2]-nonane double ring.

Some additional examples of bis-biguanides include: 1,6-bis(2-ethylhexyl diguanido hexane)dihydrofluoride; 1,6-bis(2-ethylhexyl diguanido octane)dihydrochloride; 1,6-bis(2-ethylhexyl diguanido nonane)dihydrochloride; 1,6-bis(2-ethylhexyl diguanido dodecane)dihydrochloride; or 1,6-di(4-chlorophenyl diguanido hexane)dihydrochloride or the diacetate or digluconate salt thereof.

As disclosed herein, niclosamide is effective in suppressing cancer related gene expression in cancer cell lines. For example, niclosamide is effective in suppressing EGF, insulin and/or IGF-responsive gene expression in various growth factor responsive cancer cell lines. As another example, niclosamide is effective in suppressing HIF-responsive gene expression, and furthermore, niclosamide has a potent antiproliferative effect on cancer cell lines. Accordingly, niclosamides and related compounds may be used in treatments for cancer, alone or in combination with other agents. In preferred embodiments, the cancer to be treated will be HIF-positive.

Niclosamide and related compounds (the “niclosamide class”) include salicyl anhydrides, generally, such as those described in U.S. Pat. Nos. 3,079,297 and 3,113,067. Compounds of the niclosamide class may also be selected on the basis of decreased expression of one or more HIF-responsive genes in a cancer cell line. Examples include niclosamide itself as well as bithionol (an antiinfective), dichlorophene (an antihelminthic). Further examples of members of the niclosamide class include: 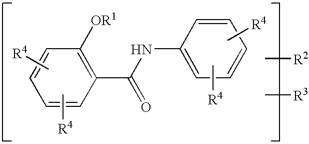
wherein R1 denotes hydrogen or an aliphatic acyl radical, R2 and R3 denote a nitro group, chlorine or bromine whereby at least R2 or R3 mean a halogen atom, R4 denotes hydrogen or halogen or alkyl or alkoxy groups with 1-5 carbon atoms or nitro groups, whereby at least one of R4 denotes a nitro group.

Niclosamides have hitherto been administered orally for the treatment of helminthic disorders. Orally administered niclosamide is poorly absorbed. Therefore, in certain embodiments, the invention provides methods for systemic or other non-oral administration of compounds of the niclosamide class. Additionally, applicants have observed that niclosamide tends to have a short serum half-life. Therefore, the invention provides formulations of compounds of the niclosamide class that are designed to provide slow release. Descriptions of various modes of administration and formulations for compounds of the niclosamide class are provided below.

In certain embodiments, the disclosure provides methods and compositions employing a cardiac glycoside. Cardiac glycosides are shown herein to be effective in suppressing cancer related gene expression in cancer cell lines. For example, cardiac glycosides are effective in suppressing EGF, insulin and/or IGF-responsive gene expression in various growth factor responsive cancer cell lines. As another example, cardiac glycosides are effective in suppressing HIF-responsive gene expression in cancer cell lines and furthermore, cardiac glycosides are shown to have potent antiproliferative effects in cancer cell lines. Accordingly, cardiac glycosides may be used in treatments for neoplastic disorders, alone or in combination with other agents.

The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorbtion and an increased half-life. Sugar moieties may be modified with one or more groups, such as an acetyl group.

In certain embodiments, the cardiac glycoside comprises a steroid core with either a pyrone substituent at C17 (the “bufadienolides form”) or a butyrolactone substituent at C17 (the “cardenolide” form).

In certain embodiments, the cardiac glycoside is represented by the general formula: 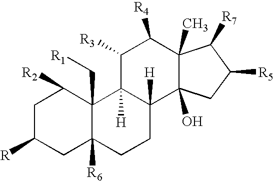
wherein
R represents a glycoside of 1 to 6 sugar residues;
R₁ represents hydrogen, —OH or ═O;
R₂, R₃, R₄, R₅, and R₆ each independently represents hydrogen or —OH;
R₇ represents

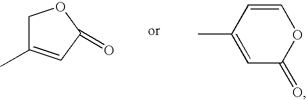

In certain preferred embodiments, the sugar residues are selected from L-rhamnose, D-glucose, D-digitoxose, D-digitalose, D-digginose, D-sarmentose, L-vallarose, and D-fructose. In certain embodiments, these sugars are in the β-conformation The sugar residues may be acetylated, e.g., to effect the lipophilic character and the kinetics of the entire glycoside. In certain preferred embodiments, the glycoside is 1-4 sugar residues in length.

A large number of cardiac glycosides are known in the art for the purpose of treating cardiovascular disorders. Given the significant number of cardiac glycosides that have proven to have anticancer effects in the assays disclosed herein, it is expected that most or all of the cardiac glycosides used for the treatment of cardiovascular disorders may also be used for treating proliferative disorders. Examples of preferred cardiac glycosides include ouabain, digitoxigenin, digoxin and lanatoside C. Additional examples of cardiac glycosides include: Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin and calotoxin. Cardiac glycosides may be evaluated for effectiveness in the treatment of cancer by a variety of methods, including, for example: evaluating the effects of a cardiac glycoside on expression of a HIF-responsive gene in a cancer cell line or evaluating the effects of a cardiac glycoside on cancer cell proliferation.

Notably, cardiac glycosides affect proliferation of cancer cell lines at a concentration well below the known toxicity level. The IC50 measured for ouabain across several different cancer cell lines ranged from about 80 nM to about 300 nM. The concentration at which a cardiac glycoside is effective as part of an antiproliferative treatment may be further decreased by combination with an additional agent that negatively regulates HIF-responsive genes, such as a redox effector or a steroid signal modulator. For example, as shown herein, the concentration at which a cardiac glycoside (ouabain) is effective for inhibiting proliferation of cancer cells is decreased 5-fold by combination with a steroid signal modulator (Casodex). Therefore, in certain embodiments, the invention provides combination therapies of cardiac glycosides with, for example, steroid signal modulators and/or redox effectors. Additionally, cardiac glycosides may be combined with radiation therapy, taking advantage of the radiosensitizing effect that many cardiac glycosides have.

In certain embodiments, the disclosure provides methods and compositions employing a redox effector. A redox effector is generally any agent that affects the redox state of the cell, often by increasing or decreasing the presence of oxygen radicals, but also be accepting or donating electrons to redox active molecules of the cell including, for example, NADH, NADPH, glutathione, and any of the various components of the mitochondrial electron transport chain. In preferred embodiments, a redox effector will cause a hypoxic cell to react as though it is, at least transiently, in a normoxic state. For example, a redox effector may inhibit HIF-1, HIF-2 or other transcription factors that are activated by hypoxia or anoxia. A redox effector may trigger degradation of HIF-1α by a ubiquitin and VHL dependent pathway. Categories of redox effectors include, for example, members of the alexidine class of compounds, members of the clofazimine class of compounds, electron transport inhibitors, cytochrome P450 inhibitors and electron acceptors.

As disclosed herein, clofazimine is effective in suppressing gene expression in various cancer cell lines. In particular, clofazimine is effective in suppressing EGF, insulin and/or IGF-responsive gene expression in various growth factor responsive cancer cell lines. Accordingly, clofazimines and related compounds may be used in treatments for neoplastic disorders, alone or in combination with other agents.

Clofazimine and related compounds (the “clofazimine class”) include compounds disclosed in U.S. Pat. No. 2,948,726. For example, compounds of the following formula: 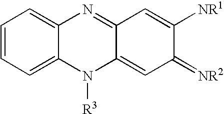
Two of the three R groups represent phenyl or the same halogenophenyl, alkylphenyl or alkoxyphenyl and the third R represents an alkyl or cycloalkyl

Compounds with known effects as inhibitors of mitochondrial electron transport (“electron transport inhibitors”) are shown herein to be effective in suppressing EGF-responsive gene expression in various EGF-responsive cancer cell lines. Accordingly, electron transport inhibitors may be used in treatments for neoplastic disorders, alone or in combination with other agents. Examples of electron transport inhibitors include members of the niclosamide class of compounds, described above.

Compounds with known effects as inhibitors of cytochrome P450 proteins (“cytochrome P450 inhibitors”) are shown herein to be effective in suppressing cancer related gene expression in cancer cell lines. In particular, cytochrome P450 inhibitors are effective in suppressing EGF, insulin and/or IGF-responsive gene expression in various growth factor responsive cancer cell lines. Accordingly, cytochrome P450 inhibitors may be used in treatments for neoplastic disorders, alone or in combination with other agents. Examples of cytochrome P450 inhibitors include proadifen and the like, such as bencyclane, cetiedil and prenylamine.

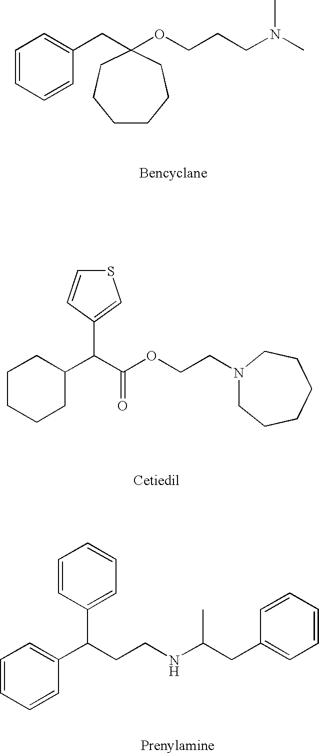

Compounds with known tendency to accept electrons from the mitochondrial electron transport chain or other physiological reducing agents (“electron acceptors”) are shown herein to be effective in suppressing cancer-related gene expression in various cancer cell lines. Accordingly, electron acceptors may be used in treatments for neoplastic disorders, alone or in combination with other agents. Examples of electron acceptors include mefloquine, chloroquine, primaquine, amodiaquin, phylloquinone, menadione (menaquinone) and the like.

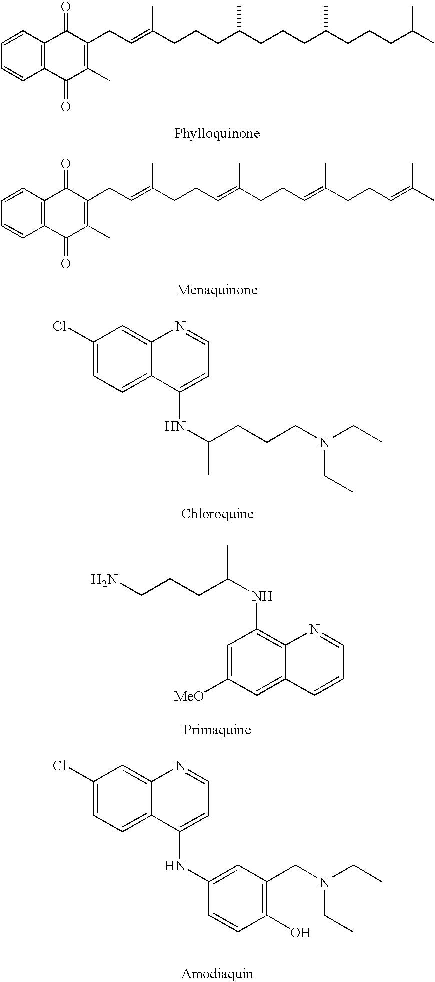

Additional regulators of HIF-1 that may be employed as redox effectors include: inhibitors of endothelin-1 (e.g. antibodies), benzoquinone ansamycins (e.g., geldanamycin), peroxides, and thioredoxin redox inhibitors (e.g., 1-methylpropyl 2-imidazolyl disulfide and pleurotin).

In certain embodiments, the disclosure provides methods and compositions employing a steroid signal modulator. A steroid signal modulator is generally any agent that affects steroid signaling, either through a nuclear or membrane bound hormone receptor. Categories of steroid signal modulators include, for example, steroid hormones generally (especially estrogens), and steroidogenesis inhibitors.

Examples of steroid hormones include estrogens such as dienestrol (see, e.g., U.S. Pat. No. 2,464,203), chlorotrianisene, colpormon, conjugated estrogenic hormones, diethylstilbestrol diphosphate, estradiol, estrone, estriol, equilin, equilenin, estradiol, estradiol benzoate, estradiol cypionate, estradiol enanthate, estradiol hemisuccinate, estradiol undecylate, estradiol valerate, estrazinol hydrobromide, estriol, estrofurate, estrone, estropipate, ethyinyl estradiol, ethinyl estradiol isopropylsulfonate, fenestrel, mestranol, moxestrol, mytatrienediol, nylestriol, polyestradiol phosphate, progesterone, quinestradiol, quinestrol. Non-steroidal estrogens include benzestrol, chlorotrianisene, clomifen, diethylstilbestrol diproprionate, dimestrol, fosfestrol, hexestrol, methestrol, and tamoxifen.

Other steroids include medroxyprogesterone acetate, levonorgestrel, gestodene, medrogestone, mestranol, estrone, hexestrol, progesterone, desogestrel, norgestimate, norethindrone, norethindone acetate, norgestrel, megestrol acetate, methyltestosterone, ethylestrenol, methandienone, oxandrolone, trimegestone, 17-hydroxyprogesterone; medroxyprogesterone; norgestrel; norethynodrel; Ethyndiol; Mestranol; Equilin; 17 alpha dihydroequilin; equilenin; 17 alpha dihydroequilenin; Leuprolide (lupron); Glucagon; Testolactone; Clomiphene; Han memopausal gonadotropins; Human chorionic gonadotropin; Urofollitropin; Bromocriptine; Gonadorelin; Luteinizing hormone releasing hormone and analogs; Gonadotropins; Danazol; Testosterone; Dehydroepiandrosterone; Androstenedione; Dihydroestosterone; Relaxin; Oxytocin; Vasopressin; Folliculostatin; Follicle regulatory protein; Gonadoctrinins; Oocyte maturation inhibitor; Insulin growth factor; Follicle Stimulating Hormone; Luteinizing hormone; Corticorelin Ovine Triffutate; Cosyntropin; Metogest; Pituitary, Posterior; Seractide Acetate; Somalapor; Somatrem; Somatropin; Somenopor; Somidobove.
Examples of Estrogens include:

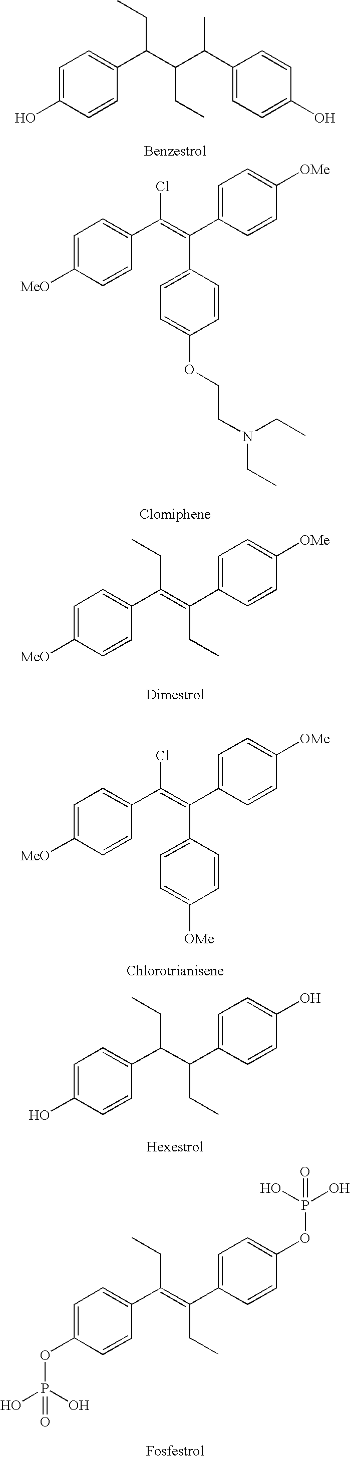

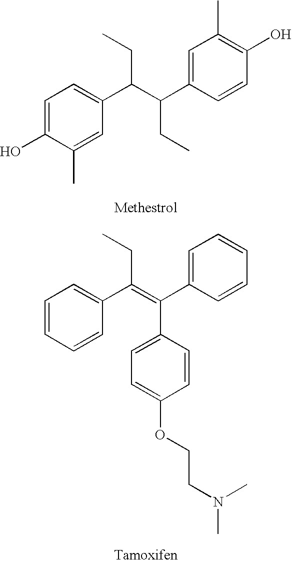

Steroidogenesis inhibitors are shown herein to be effective in suppressing cancer related gene expression in cancer cell lines. In particular, steroidogenesis inhibitors are effective in suppressing EGF, insulin and/or IGF-responsive gene expression in various growth factor responsive cancer cell lines. Accordingly, steroidogenesis inhibitors may be used in treatments for neoplastic disorders, alone or in combination with other agents. Preferred steroidogenesis inhibitors include bicalutamide, clotrimazole and econazole. Bicalutamide and related compounds are described, for example, in U.S. Pat. No. 4,636,505, directed to Amide Derivatives. Examples of steroidogenesis inhibitors including, and related to bicalutamide include amide derivatives generally as shown below: 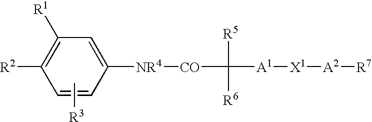
where A¹ is alklene of up to six carbon atoms; A² is a direct link or alkylene of up to six carbon atoms; R¹ is cyano, nitro, halogeno, alkyl, alkoxy, alkanoyl, alkylthio, alkylsulphinyl, alkylsulphonyl, or perfluoroalkyl each of up to four carbon atoms; R² is is cyano, nitro, halogeno, alkanoyl, alkylthio, alkylsulphinyl, alkylsulphonyl, or perfluoroalkyl each of up to four carbon atoms; R³ is hydrogen or halogen, R⁴ is hydrogen or alkyl up to four carbon atoms; R⁵ is hydroxyl, alkoxy, acyloxy, each of up to fifteen carbon atoms; R⁶ is alkyl or halogenoalkyl of up to four carbon atoms; R⁷ is alkyl, alkenyl, hydroxyalkyl or cycloalkyl, each up to six carbon atoms, or R⁷ is phenyl which bears one or two substituents selected from hydrogen, halogen, nitro, cyano, alkyl, alkoxy, alkanoyl, alkylthio, alkylsulphinyl, alkylsulphonyl, and perfluoroalkyl, or R⁷ is naphthyl, or R⁷ is a 5- or 6-membered saturated or unsaturated heterocyclic which contains one, two, or three heteroatoms selected from oxygen, nitrogen, and sulphur, which hterocyclic may be a single ring or may be fused to a benzo-ring, and which hterocyclic is unsubstituted or bears one or two halogen or alkyl of up to four carbon atoms substituents; and X¹ is sulphur, sulphinyl, or sulphonyl.

Other examples of steroidogenesis inhibitors include the N-trityl-imidazoles generally as shown below: 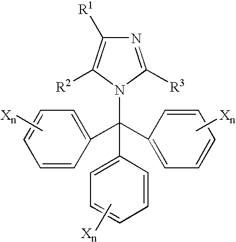
where R¹, R² and R³ are hydrogen, lower alkyl or phenyl or R¹ and R² together form an anellated benzene ring, X, X′ and X″ are alkyl of 1 to 12 carbon atoms or an electo-negative moiety, and n, n′ and n″ are an integer from 0 to 2. When

Other related compounds include metronidazole, nitroimidazole and miconazole. 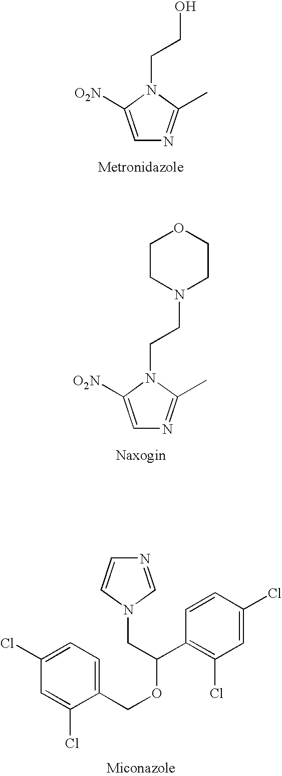
Econazole and related compounds are described, for example, in U.S. Pat. No. 3,717,655, directed to 1-(β-Aryl)Ethyl-Imidazole Derivatives. An example of econazole structure is below: 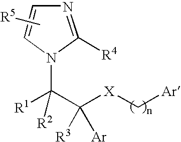
where R, R¹ and R² are each a member selected from the group consisting of hydrogen and lower alkyl; X is a member selected from the group consisting of oxy and NH1; n is an integer zero, 1 or 2; Ar is a member selected from the group consisting of phenyl, substituted phenyl, thienyl and halothienyl, said substituted phenyl containing at least one phenyl substitutent selected from the group consisting of halo, lower alkyl and lower alkoxy; Ar′ is a member selected from the group consisting of phenyl, substituted phenyl and alpha-tetralyl, said substituted phenyl containing at least one phenyl substituent selected from the group consisting of halo, lower alkyl, lower alkoxy, cyano, nitro and amino; R′ is a member selected from the group consisting of hydrogen, methyl and ethyl; and R″ is a member selected from the group consisting of hydrogen and methyl.

In certain embodiments, the disclosure provides methods and compositions employing a growth factor antagonist, particularly an EGF, IGF or insulin antagonist. A growth factor antagonist, as the phrase is used herein, is intended to include only those antagonists having a proximal effect on the growth factor, its receptor or immediate downstream signaling events. Examples include antibodies and soluble receptor fragments, or other reagents that bind specifically to the targeted growth factor and inhibit its activity. Other examples include agents that inhibit receptor kinase activity. For example, tyrphostins such as AG1024 and AG1478 may be used as EGF and insulin antagonists, respectively. RNAi constructs that inhibit expression of the growth factor receptor, the growth factor itself or proximal signaling proteins may also be used. Such constructs may be in the form of short double stranded RNAs (or RNA:DNA hybrids, so long as the antisense strand is RNA) or in the form of single polynucleotides that fold to form a short “hairpin” region of double helix. Other examples of EGF antagonists that act as tyrosine kinase inhibitors include anilinoquinazolines such as gefitinib (Iressa®), with the chemical name 4-Quinazolinamine, N-(3-chloro4-fluorophenyl)-7-methoxy-6-[3-4-morpholin)propoxy], erlotinib(4-(3-ethynylphenylamino)-6,7-bis(2-methoxyethoxy)quinazoline).

In certain embodiments, the various compounds described herein can be used alone. Alternatively, the subject compounds may be used in combination with each other or with conventional anti-cancer therapeutic approaches directed to treatment or prevention of proliferative disorders. For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject therapeutic agents.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When an anti-neoplastic agent of the present invention is administered in combination with another conventional anti-neoplastic agent, either concomitantly or sequentially, such therapeutic agent is shown to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine. Bicalutamide and campothecin are preferred for use in combination with cardiac glycosides and redox effectors.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

3. Pharmaceutical Formulations and Administration

Any of the various agents and combinations disclosed herein may be formulated for use as a pharmaceutical. Such formulations may be designed for systemic (e.g. venous) administration or other delivery means, as appropriate, such as topical, rectal or vaginal delivery. Pharmaceutical compositions for use in accordance with the present invention may be formulated in conventional manner using one or more physiologically acceptable carriers (the term “carrier” is intended to mean any agent that is included for purposes other than an anti-neoplastic effect, including, for example, buffers, solvents, permeabilizers, salts, material to which the agent is adsorbed, etc.). The therapeutic compositions of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the therapeutic compositions of the invention can be formulated in liquid solutions, preferably in physiologically compatible buffers such as Hank's solution or Ringer's solution. In addition, the therapeutic compositions may be formulated in solid form and redissolved or suspended immediately prior to use. Lyophilized forms are also included.

The therapeutic compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. Niclosamide, for example, is conventionally administered orally, however, in certain preferred embodiments, niclosamide can be formulated for systemic administration.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compositions of the invention are formulated into ointments, salves, gels, or creams as generally known in the art. A wash solution can be used locally to treat an injury or inflammation to accelerate healing.

For oral administration, the therapeutic compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active agent. For buccal administration the therapeutic compositions may take the form of tablets or lozenges formulated in conventional manner. For administration by inhalation, the compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the therapeutic agents and a suitable powder base such as lactose or starch.

In some instances it will be desirable to provide a slow release formulation for a compound disclosed herein, particularly in the case of compounds that have short serum half-lives, such as niclosamide. Accordingly therapeutic compositions may be formulated as a depot preparation. Such long acting formulations may be administered by implantation (for example subcutaneously or intramuscularly) or by intramuscular injection. Thus, for example, the therapeutic compositions may be formulated with suitable polymeric or hydrophobic materials (for example as an emulsion in an acceptable oil) or ion exchange resins, or as sparingly soluble derivatives, for example, as a sparingly soluble salt. Art known slow release formulations and/or carriers, e.g., emulsions, liposomes, gels, suspensions, biocompatible polymers and gum matrices may be employed. Slow release formulations according to the invention also include, e.g., specially coated pellets, polymer formulations or matrices for surgical insertion or controlled release microparticles or microspheres for implantation, insertion or injection, wherein the slow release of the active medicament is brought about through controlled diffusion out of the formulation and/or selective breakdown of the coating of the preparation, or selective breakdown of a polymer matrix, if present.

The controlled release material should be biocompatible and be degraded, dissolved or absorbed in situ in a safe and pharmaceutically acceptable manner so that the material is removed from the injection or implantation site by natural tissue processes and in a suitable amount of time, e.g., less than one year, preferably less than 6 months, and most preferably in less than one month. In any event, the carrier may be removed by local tissue processes without causing untoward local tissue reaction, or systemic or local toxicity. In the case of polymeric materials, biocompatibility is enhanced by preparing the polymeric materials in a pharmaceutically acceptable manner, i.e., employing purification techniques well known to the art, e.g., recrystallization of either the monomers forming the polymer and/or the polymer and other techniques for producing materials for implantation or injection into living tissue.

Suitable biodegradable polymers can be utilized as the controlled release material. The polymeric material may comprise a polylactide, a polyglycolide, a poly(lactide-co-glycolide), a polyanhydride, a polyorthoester, polycaprolactones, polyphosphazenes, polysaccharides, proteinaceous polymers, soluble derivatives of polysaccharides, soluble derivatives of proteinaceous polymers, polypeptides, polyesters, and polyorthoesters. The polysaccharides may be poly-1,4-glucans, e.g., starch glycogen, amylose, amylopectin, and mixtures thereof. The biodegradable hydrophilic or hydrophobic polymer may be a water-soluble derivative of a poly-1,4-glucan, including hydrolyzed amylopectin, hydroxyalkyl derivatives of hydrolyzed amylopectin such as hydroxyethyl starch (HES), hydroxyethyl amylose, dialdehyde starch, and the like. Preferred controlled release materials which are useful in the formulations of the invention include the polyanhydrides, co-polymers of lactic acid and glycolic acid wherein the weight ratio of lactic acid to glycolic acid is no more than 4:1 (i.e., 80% or less lactic acid to 20% or more glycolic acid by weight), and polyorthoesters containing a catalyst or degradation enhancing compound, for example, containing at least 1% by weight anhydride catalyst such as maleic anhydride. Other useful polymers include protein polymers such as gelatin and fibrin and polysaccharides such as hyaluronic acid. Since polylactic acid takes at least one year to degrade in vivo, this polymer should be utilized by itself only in circumstances where such a degradation rate is desirable or acceptable.

Commercially available polymers include, for example, poly(d,1-lactic-co-glycolic acid) and preferrably 50:50 poly (D,L) lactic co-glycolic acid having a mole percent composition of 50% lactide and 50% glycolide. Other suitable commercially available products include 65:35 DL, 75:25 DL, 85:15 DL and poly(d,1-lactic acid) (d,1-PLA). Poly(lactide-co-glycolides) are commercially available, for example, from Boerhinger Ingelheim (Germany) under its Resomer. mark, e.g., PLGA 50:50 (Resomer RG 502), PLGA 75:25 (Resomer RG 752) and d,1-PLA (resomer RG 206), and from Birmingham Polymers (Birmingham, Ala.). These copolymers are available in a wide range of molecular weights and ratios of lactic to glycolic acid.

Proteinaceous polymers, may also be used. Proteinaceous polymers and their soluble derivatives include gelation biodegradable synthetic polypeptides, elastin, alkylated collagen, to alkylated elastin, and the like. Biodegradable synthetic polypeptides include poly-(N-hydroxyalkyl)-L-asparagine, poly-(N-hydroxyalkyl)-L-glutamine, copolymers of N-hydroxyalkyl-L-asparagine and N-hydroxyalkyl-L-glutamine with other amino acids. Suggested amino acids include L-alamine, L-lysine, L-phenylalanine, L-valine, L-tyrosine, and the like.

In embodiments where the biodegradable polymer comprises a gel, one such useful polymer is a thermally gelling polymer, e.g., polyethylene oxide, polypropylene oxide (PEO-PPO) block copolymer such as Pluronic™ F127 from BASF Wyandotte. In such cases, the formulation may be injected via syringe as a free-flowing liquid, which gels rapidly above 30° C. (e.g., when injected into a patient). The gel system then releases a steady dose of active ingredient at the site of administration.

In additional embodiments of the invention, the controlled release material, which in effect acts as a carrier for the local anesthetic, can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan. Further, the pharmacokinetic release profile of these formulations may be first order, zero order, bi- or multi-phasic, to provide the desired effect over the desired time period.

Definitions or further descriptions of any of the foregoing terminology are well known in the art and may be found by referring to any standard biochemistry reference text such as “Biochemistry” by Albert L. Lehninger, Worth Publishers, Inc. and “Biochemistry” by Lubert Stryer, W.H. Freeman and Company, both of which are hereby incorporated by reference.

The aforementioned biodegradable hydrophobic and hydrophilic polymers are particularly suited for the methods and compositions of the present invention by reason of their characteristically low human toxicity and virtually complete biodegradability.

A desired release profile can be achieved by using a mixture of polymers having different release rates and/or different percent loading of local anesthetic and/or augmenting agent, for example, polymers releasing in one day, three days, and one week, so that linear release is achieved even when each polymer per se does not release linearly over the same time period. In addition, a mixture of microspheres having one or more different antineoplastic agents, having the same or different controlled release profile, can be utilized to provide the benefits of different potencies and spectrum of activity during the course of treatment. In other embodiments of the invention, the controlled release material comprises an artificial lipid vesicle, or liposome. Liposomes are well known in the art as carriers of bioactive or pharmacologically active substances such as drugs. A wide variety of lipid materials may be used to form the liposomes including natural lecithins, e.g., those derived from egg and soya bean, and synthetic lecithins, the proviso being that it is preferred that the lipids are non-immunogenic and biodegradable. Also, lipid-based materials formed in combination with polymers may be used, such as those described in U.S. Pat. No. 5,188,837 to Domb, (incorporated by reference herein).

In other preferred formulations, the lipids containing the neoplastic agent are dispersed in a pharmaceutically acceptable aqueous medium. In a further embodiment, a portion of the dose of the antineoplastic agent is incorporated into the aqueous medium in immediate release form to form an aqueous pharmaceutical suspension useful for administration at the desired site in the patient.

The therapeutic compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Toxicity and therapeutic efficacy of compositions of the present invention can be determined by standard pharmaceutical procedures in cell cultures or experimental animals, e.g., for determining The LD50 (The Dose Lethal To 50% Of The Population) and The Ed50 (the dose therapeutically effective in 50% of the population). The dose ratio between toxic and therapeutic effects is the therapeutic index and it can be expressed as the ratio LD50/ED50. Therapeutic agents which exhibit large therapeutic indices are preferred. While therapeutic compositions that exhibit toxic side effects may be used, care should be taken to design a delivery system that targets such therapeutic agents to the site of affected tissue in order to minimize potential damage to uninfected cells and, thereby, reduce side effects.

The data obtained from the cell culture assays and animal studies can be used in formulating a range of dosage for use in humans. The dosage lies preferably within a range of circulating concentrations that include the ED50 with little or no toxicity. The dosage may vary within this range depending upon the dosage form employed and the route of administration utilized. For any agents used in the method of the invention, the therapeutically effective dose can be estimated initially from cell culture assays. A dose may be formulated in animal models to achieve a circulating plasma concentration range that includes the IC50 (i.e., the concentration of the test therapeutic agent which achieves a half-maximal inhibition of symptoms) as determined in cell culture. Such information can be used to more accurately determine useful doses in humans. Levels in plasma may be measured, for example, by high performance liquid chromatography.

In a preferred embodiment, cardiac glycosides are administered to a patient by using osmotic pumps, such as Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, ALZET's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 μL/hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

4. Cells and Assays

In yet a further aspect, the invention relates to the discovery of cells comprising a reporter gene and/or one or more selectable markers that are expressed in response to extracellular signaling. The phrase “extracellular signaling” is intended to refer any relatively undefined effects that cells have on themselves or on neighboring cells in a culture through release of one or more secreted factor (e.g. autocrine or paracrine signaling). The phrase is also intended to encompass cell-cell signaling that occurs in response to contacts between one or more membrane bound effectors, such as integrins, eph/ephrins, and cadherins. An assay that focuses on only one or a few predetermined signaling molecules is not encompassed by the term “extracellular signaling”. In certain aspects, the power of assessing responsiveness to general extracellular signaling events is the ability to identify previously unrecognized signaling responsive elements of the cell.

Whether a gene or other cellular characteristic is responsive to extracellular signaling may be determined in a variety of ways. For example, expression of a gene (or other characteristic) may be measured in relatively dense cell cultures and compared to that in relatively sparse cell cultures. As another example, a characteristic may be measured in relatively sparse cell cultures, where one culture is treated with medium conditioned by a dense cell culture, while the other culture is not so treated. Likewise, various flow systems or transwell systems may be designed to assess extracellular signaling effects.

Assay systems designed to assess responsiveness to extracellular signaling may be used to identify agents and genes that participate in or affect a variety of processes, including neoplasia, stromal signaling, stem cell regulation, wound healing, etc.

In certain embodiments, the disclosure provides cells comprising: a nucleic acid construct which includes one or more of the following: a positive selection marker, a negative selection marker and a reporter gene, wherein expression of the reporter and or selection markers are regulated by extracellular signaling. In a preferred embodiment, the construct comprises both a positive selection marker and a negative selection marker, and optionally a reporter gene. Whether a selectable marker or reporter gene is responsive to extracellular signaling may be characterized in a variety of ways, including, for example, a comparison of expression in a high cell density cell culture and in a low cell density cell culture, where a responsive gene will be expressed at a higher level in the high cell density cell culture. Optionally, expression of the marker(s) and/or reporter gene are regulated by a hypoxia-responsive transcription factor, such as HIF-1 or HIF-2. Optionally, expression of the marker(s) and/or reporter gene are inhibited by an EGF antagonist or a compound such as a redox effector or steroid signaling modulator.

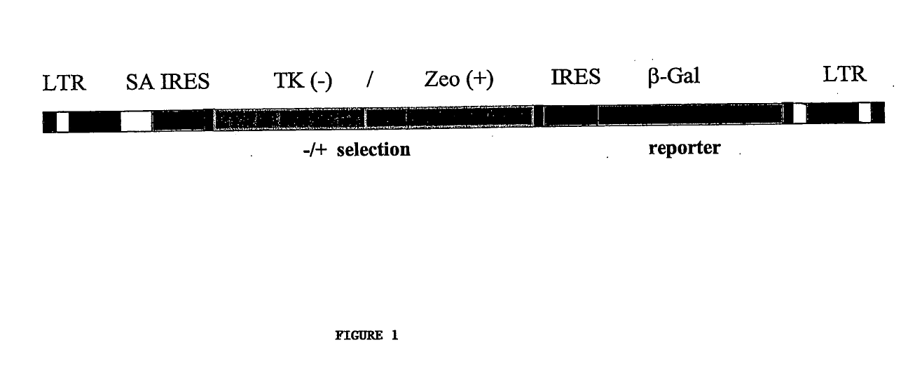

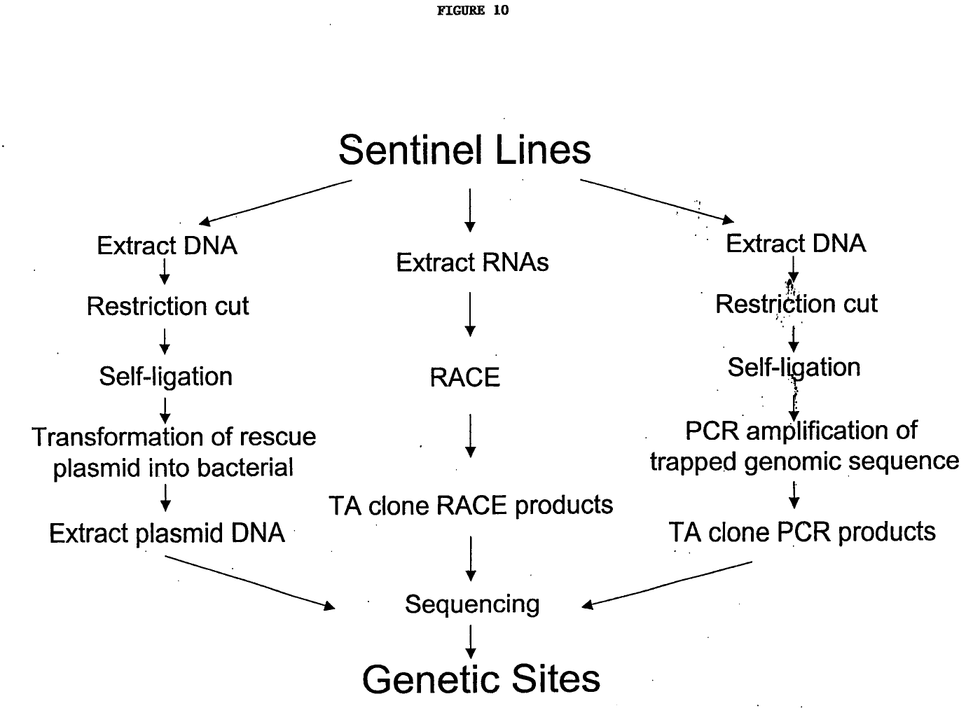

Examples of nucleic acid constructs and systems for inserting same into cells may be found, for example, in the published U.S. patent Publication 20030003519 A1, published Jan. 2, 2003, incorporated by reference herein. An exemplary construct is shown in FIG. 1. In this construct, a basal promoter operably linked to a positive selection marker, a negative selection marker and a reporter gene. If this construct is inserted into the genome, it may become regulated by endogenous cis-acting regulatory elements. The result will be a cell line carrying a positive selection marker, a negative selection marker and a reporter gene all coordinately regulated by an endogenous transcriptional regulatory system. According to the methods disclosed herein, it is possible to identify those cells in which the nucleic acid construct is responsive to extracellular signaling. For example, cells may be cultured in conditions of high extracellular signaling and subjected to the positive selection marker, which eliminates those cells that do not express the marker in conditions of high extracellular signaling. The cells may then be cultured in conditions of low extracellular signaling and exposed to the negative selection marker, thus eliminating those cells that express the markers in the absence of significant extracellular signaling. Responsiveness to extracellular signaling may be verified and quantified by selecting individual cell lines that survive the preceding treatment, and measuring reporter gene expression in a variety of different conditions. In this manner, it is possible to rapidly develop cell lines containing a reporter gene that is responsive to extracellular signaling. In general, such an insert is expected to show transcriptional regulation that is similar to the gene that is normally at that locus in the genome. The insert may be used to identify the endogenous regulated gene (see, for example, FIG. 10), giving insight into the molecular process that is so regulated. Genes identified in this manner may, in themselves, be suitable targets for drug screening assays. In addition, the extracellular signaling responsive cell lines may be used to identify compounds that modify the expression of the responsive reporter gene. For cell types in which extracellular signaling is known to be particularly important (e.g., cancers, stem cells), compounds that modulate expression of the responsive gene are likely to be suitable for modulating the corresponding process in vivo.

In certain embodiments, the disclosure provides methods for identifying candidate anti-neoplastic agents, the method comprising: (a) culturing a cell comprising an extracellular signaling responsive reporter gene under conditions wherein the reporter gene is detectably expressed; (b) contacting the cell with a test agent; (c) detecting expression of the reporter gene; and (d) comparing the expression of the reporter gene in (c) to an untreated reference, wherein a test agent that causes a decrease in the expression of the reporter gene relative to the untreated reference is a candidate anti-neoplastic agent. A reference may be, for example, a simultaneous untreated control cell or any record of such previously measured control cell. Optionally, the test agent may be selected from among the following: a growth factor antagonist, a redox effector and a steroid signaling modulator. In a preferred embodiment, the cell is derived from an established cancer cell line, such as an established pancreatic cancer cell line (e.g. Panc1)or a non-small cell lung carcinoma cell line (e.g. A549).

In certain embodiments, the disclosure provides methods for identifying a candidate anti-neoplastic agent, the method comprising: (a) contacting a cell with a test agent, wherein the cell comprises a reporter gene that is responsive to extracellular signaling; (b) detecting expression of the reporter gene; and (c) comparing the expression of the reporter gene in (b) to an untreated reference, wherein a test agent that causes a decrease in the expression of the reporter gene relative to the untreated reference is a candidate anti-neoplastic agent.

In further embodiments, the disclosure provides methods for identifying effective anti-neoplastic combinations, the method comprising: (a) culturing a cell comprising an extracellular signaling responsive reporter gene under conditions wherein the reporter gene is detectably expressed; (b) contacting the cell with a first candidate anti-neoplastic agent and a second candidate anti-neoplastic agent; (c) detecting expression of the reporter gene; and (d) comparing the expression of the reporter gene in (c) to an appropriate reference, wherein, a combination of first candidate anti-neoplastic agent and second candidate anti-neoplastic agent that decreases expression of the reporter gene more than either anti-neoplastic agent alone is an effective anti-neoplastic combination.

In certain embodiments, the disclosure provides methods of generating cancer cells expressing a reporter gene that is responsive to extracellular cell signaling, the method comprising: (a) transfecting a cancer cell line with a nucleic acid construct including a positive selection marker, a negative selection marker and a reporter gene, wherein expression of the positive selection marker, the negative selection marker and the reporter gene is coordinately regulated; (b) culturing the transformants to a high cell density; (c) selecting for cells expressing the negative selection marker, thereby eliminating a substantial portion of the cultured cells; and (d) selecting for cells that do not express the positive selection marker, wherein cells selected in (d) are cancer cells that express a reporter gene in response to extracellular cell signaling.

Test agents may be selected from libraries of compounds, and may include, for example, small molecules, peptides, RNAi constructs, antibodies, natural products, or any other desired compounds.

In one embodiment a nucleic acid construct for use in preparing a vector for generating a panel of cells comprises the following elements in downstream (5′ to 3′) sequence: a cassette containing an internal ribosome entry site; a transactivator polypeptide coding sequence encoding a polypeptide, said polypeptide acting as a regulator unit to one or more regulatory elements contained in a genomic loci in a particular cell or cell type of interest, said transactivator polypeptide being responsive to one or more regulatory elements contained in a genomic loci in a cell of interest; a translation stop sequence; an internal ribosome entry site; a reporter element responsive to at least one stimulatory agent; and a translation stop sequence.

In preferred features of this embodiment of the invention, the reporter element can be an enzyme, such as secreted alkaline phosphatase, Luciferase™, or green fluorescent protein (“GFP”), the marker polypeptide can be a the promoterless protein coding sequence, such as a tetracycline regulator unit (tTA). A nucleic acid cassette containing these elements can be incorporated into an induction gene trap vector containing a splice acceptor site; an internal ribosome entry site; a marker polypeptide coding sequence encoding a polypeptide providing selection traits and being responsive to one or more regulatory elements contained in a genomic loci in a particular cell or cell type of interest; and a translation stop sequence. The marker polypeptide can be a fusion protein with positive and negative selection traits. Negative selection traits can be provided in situations whereby the expressed gene leads to the elimination of the host cell, frequently in the presence of a nucleoside analog, such as gancyclovir. Positive selection traits can be provided by drug resistance genes. Suitable negative selection markers include, for example, DNA sequences encoding Hprt, gpt, HSV-tk, diphtheria toxin, ricin toxin, and cytosine deeaminase. Suitable positive selection traits include, for example, DNA sequences encoding neomycin resistance, hygromycin resistance, histidinol resistance, xanthine utilization, Zeocin resistance, and bleomycin resistance. A particularly preferred fusion protein is a fusion protein encoding Tk-Zeo.

This nucleic acid construct can be incorporated into a vector, such as a viral vector, and preferably a retroviral vector, to transfect cells of interest. This can be accomplished by introducing the vector into a medium containing the cells using techniques known to those skilled in the art. Suitable techniques are described in U.S. Pat. No. 5,922,601, the disclosure of which is incorporated herein by reference in its entirety. Not all of the cells will be successfully transfected, meaning that the vector will not be integrated into the genomic loci of the cell. Successful integration events can be selected for using a drug selection compound, such as zeocin. If the vector contains a zeocin resistant gene, the zeocin will serve to kill the cells in which the vector has not been successfully integrated into the genome of the cell.

Once cells which have been successfully transfected, and the vector has been operably integrated into the genome of the cell, the cells may be selected for activity with respect to specific conditions, such as high levels of extracellular signaling. Such activity can include those cells in which regulatory factors, such as enhancers and promoters, have been turned on by the extracellular signaling, and those cells in which the appropriate regulatory factors have been turned off by extracellular signaling, as described above, an, in U.S. patent Publication 20030003519 A1.

The sequence of the trapped regulatory elements upstream of the integrated construct may be determined using standard tehniques, as illustrated in FIG. 10. Additionally, the coding sequence for the trapped gene (mRNA) that is upstream and/or downstream of the integrated construct may be determined using standard cDNA amplification and sequencing methods.

The application will be more readily understood by reference to the following examples, which are included merely for purposes of illustration of certain aspects and embodiments of the present application, and are not intended to limit the application.

### EXAMPLES

### Example 1

### Identification of Cell Lines that Are Responsive To Extracellular Signaling

Cancer cell lines (Panc1, for pancreatic cancer, and A549 for non-small cell lung carcinoma) were transfected with the construct depicted in FIG. 1. The retroviral vector contained a thymidylate kinase gene (a negative selection marker), a zeocin resistance marker (a positive selection marker) and a gene encoding beta-galactosidase “β-gal”, as a reporter gene.

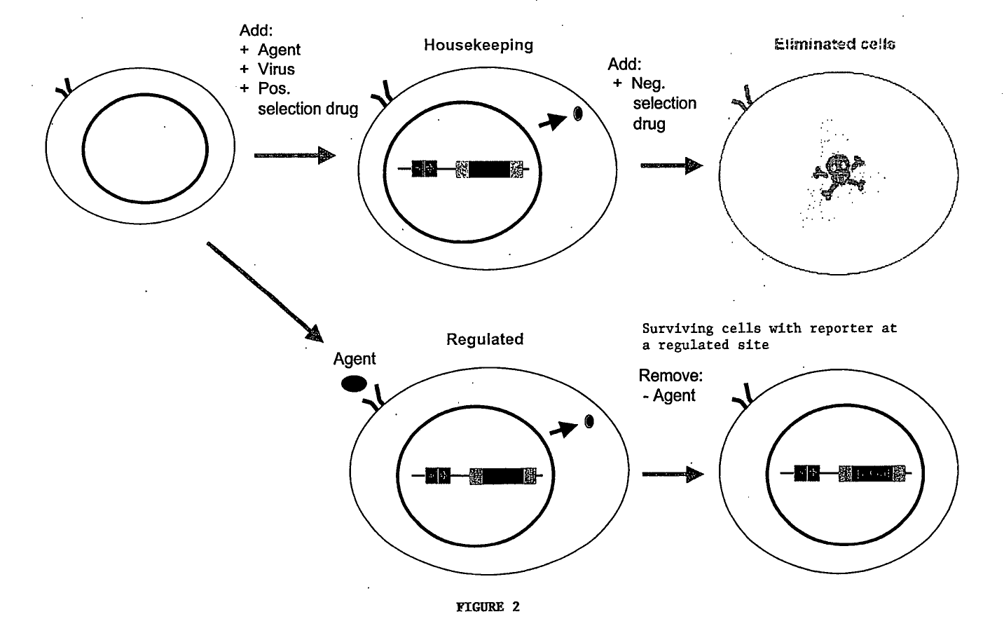

Cells were grown to a significant cell density and exposed to zeocin, so as to select for cells expressing the zeocin marker in the presence of high levels of extracellular signaling. Cells were also exposed to a growth factor, such as EGF, insulin or IGF, although in some instances, the growth factor had only minor effects on gene expression compared to the generalized effect of extracellular signaling. The selected cell cultures were greatly depleted of cells by zeocin treatment, as the vast majority of cells were eliminated by the zeocin. Therefore, after zeocin selection, the level of extracellular signaling was greatly reduced. Under these conditions, cells were exposed to an agent such as ganciclovir that causes lethality in TK+ cells. This selection step eliminated those cells containing a nucleic acid construct that is expressed at significant levels even in low extracellular signaling conditions. The resulting cells were then used for further study. See FIG. 2.

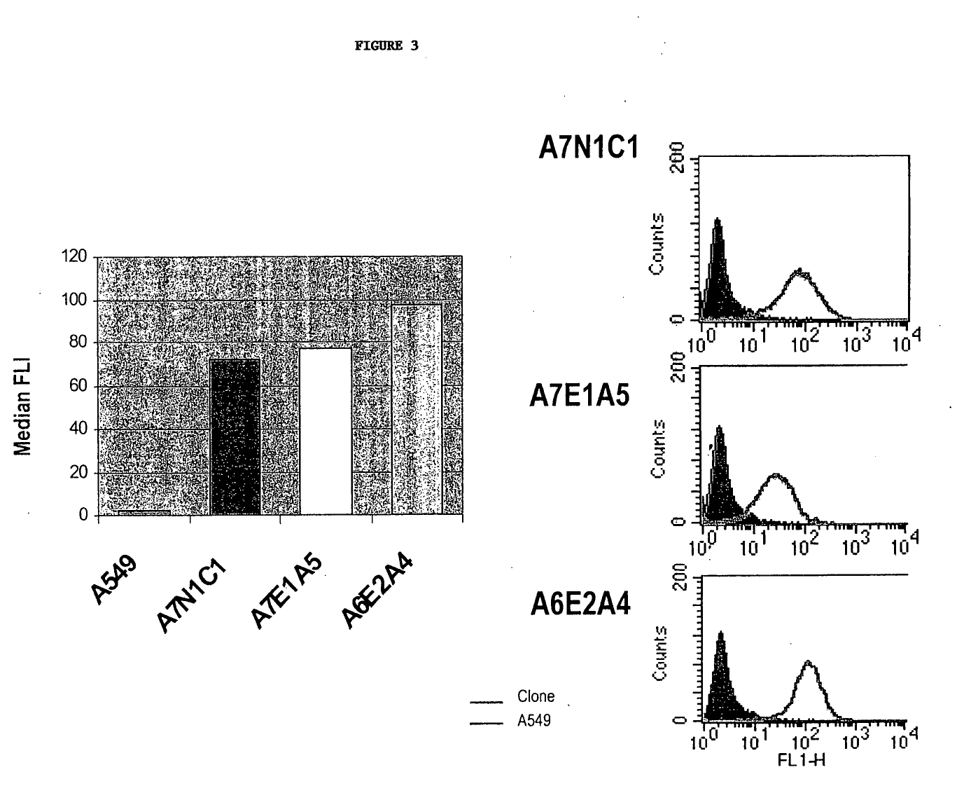

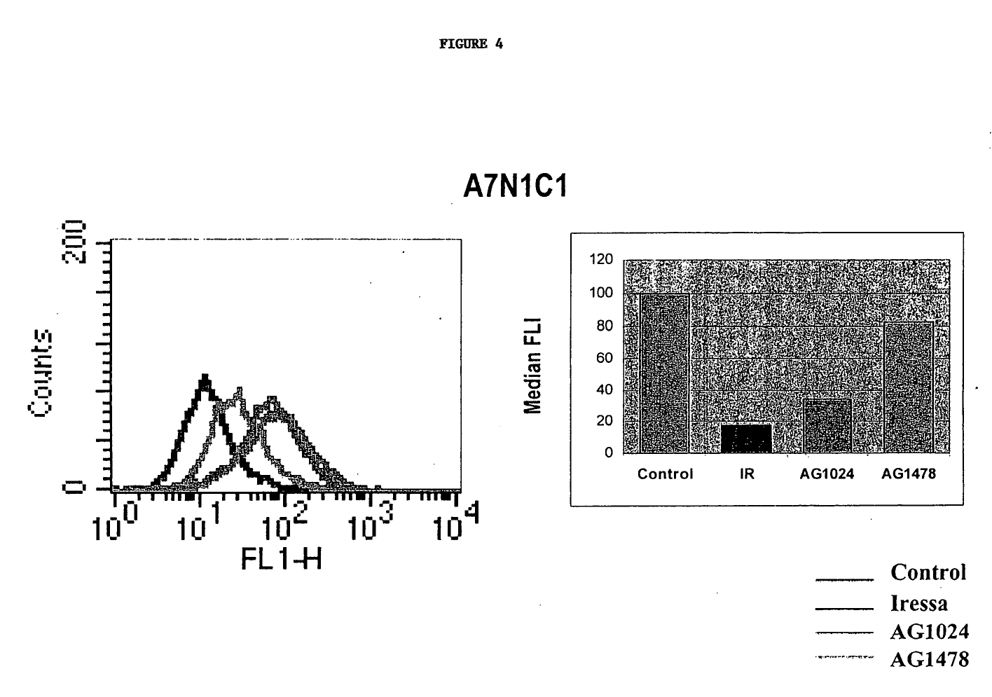

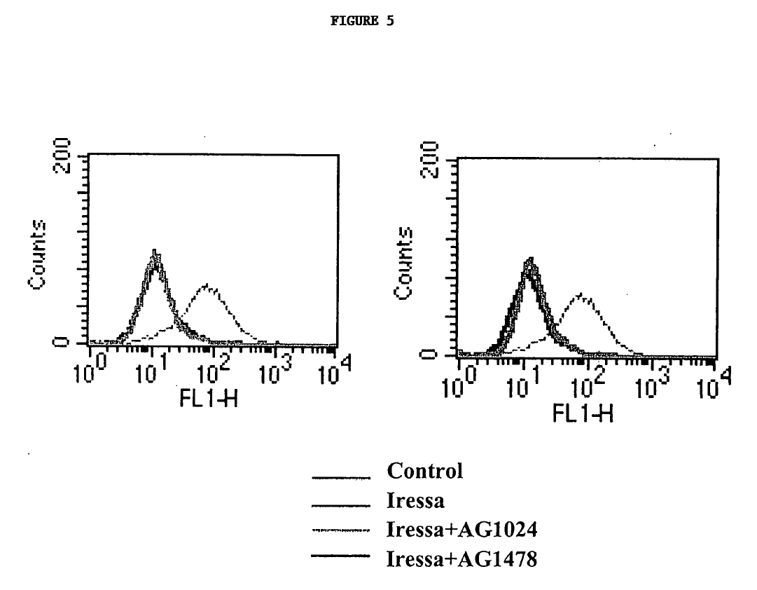

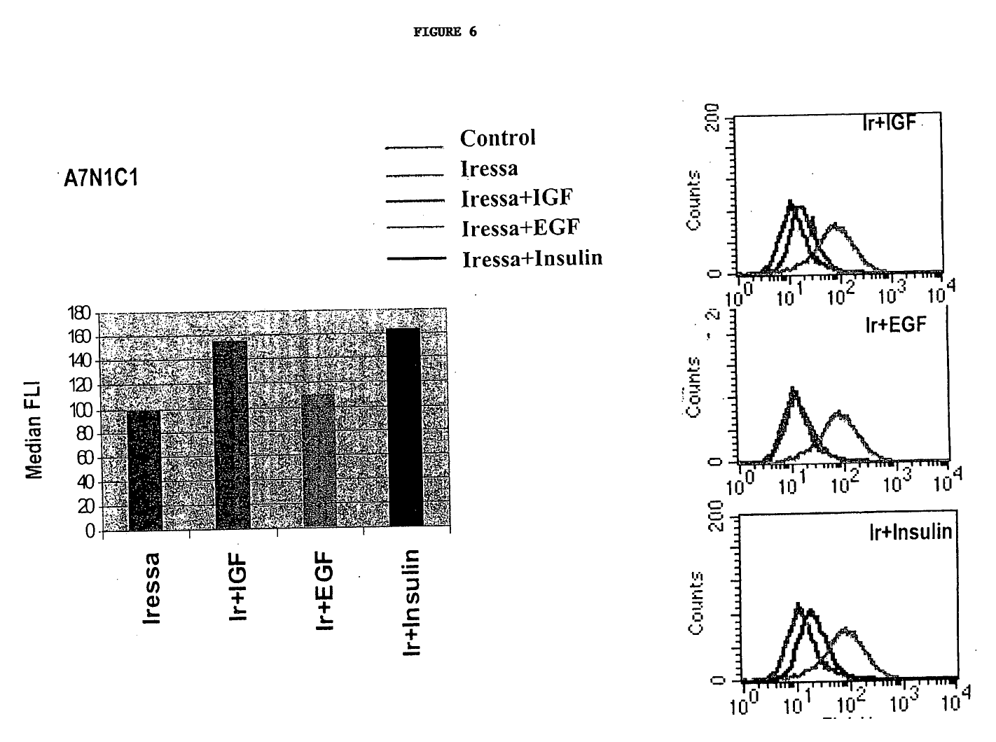

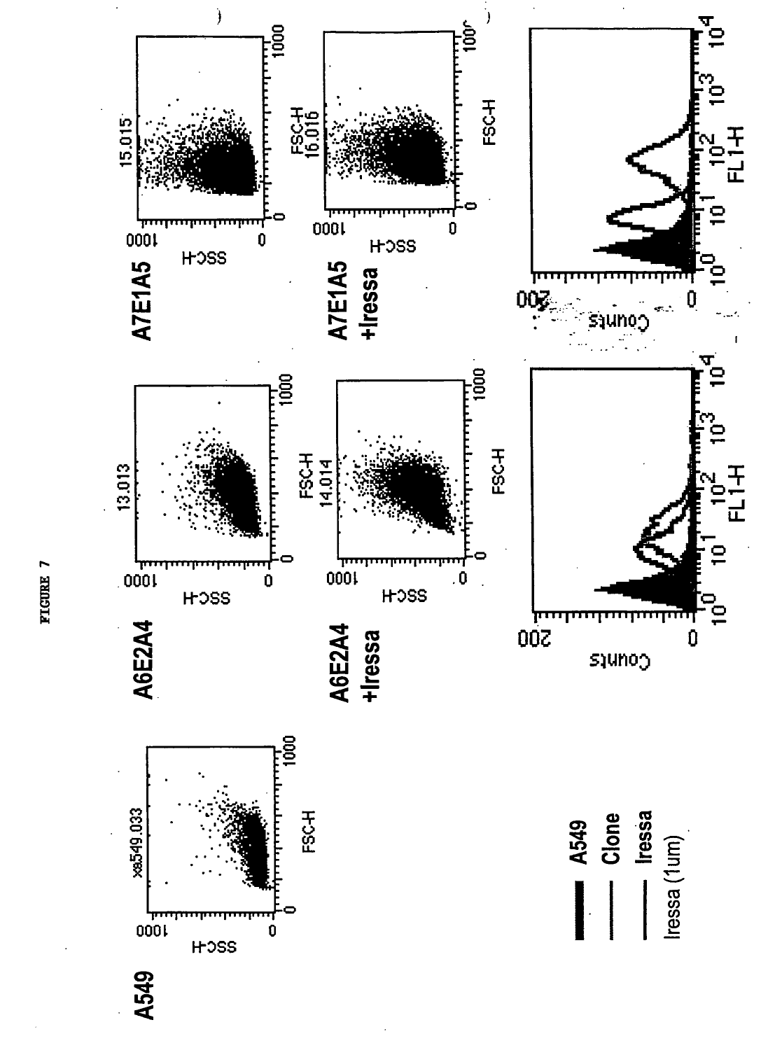

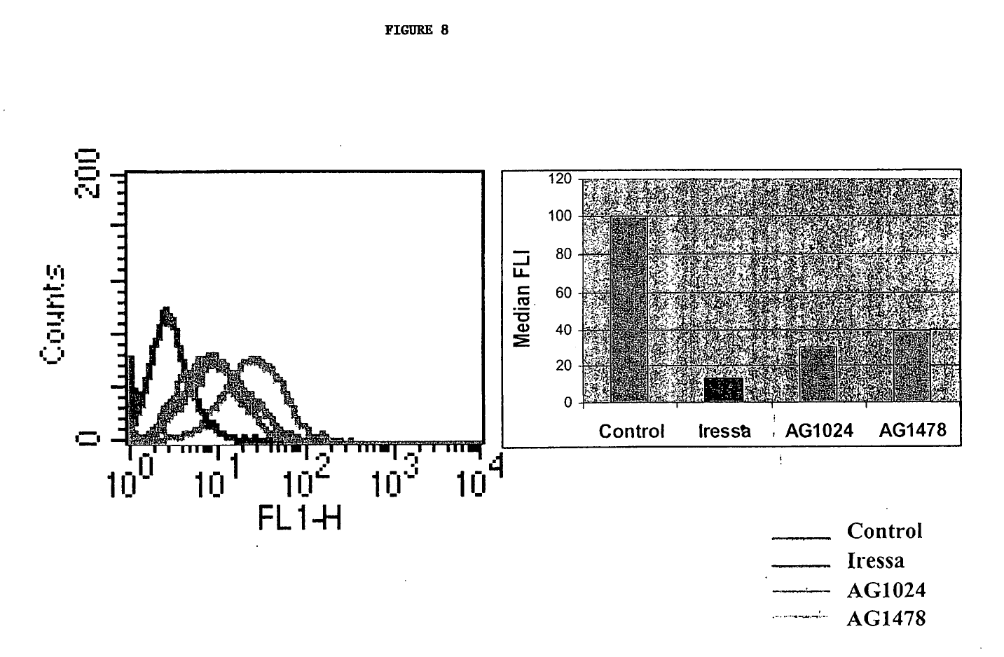

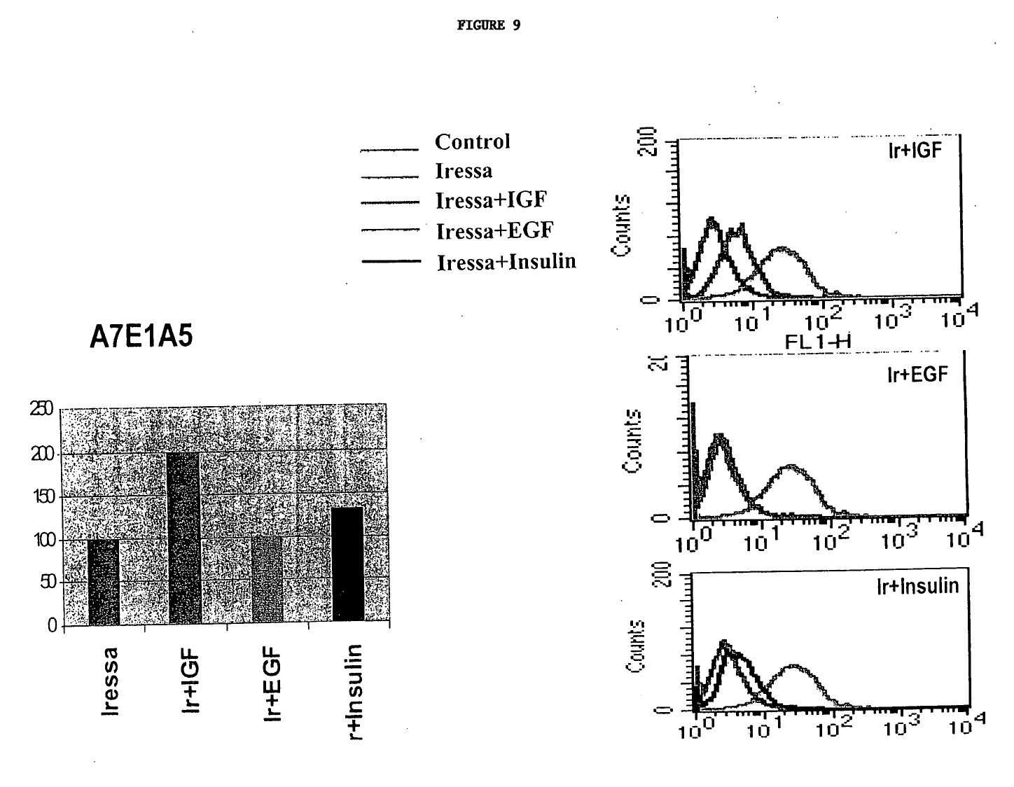

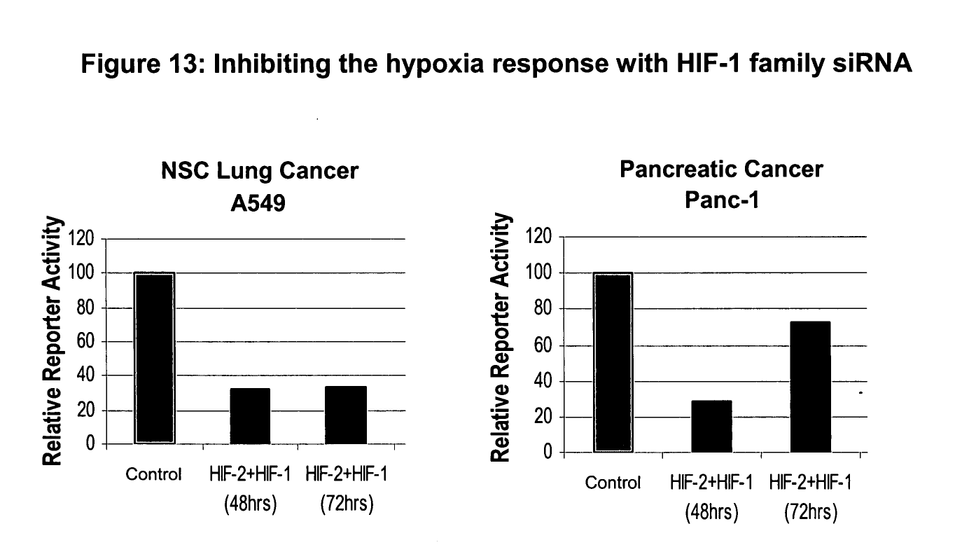

FIGS. 3 through 9 depict the results from a series of experiments characterizing the recovered cell lines. These experiments confirmed that EGF-responsive gene expression is inhibited by an EGF antagonist, Iressa, as well as a tyrphostin EGF antagonist. These experiments also show that in cell lines that are responsive to extracellular signaling, insulin and EGF antagonists affect gene expression. In data shown in FIG. 13 with respect to certain cell lines, RNAi knockdowns of the HIF-1 and HIF-2 transcription factors led to decreased gene expression, demonstrating that the recovered cell lines contained inserts at positioned regulated both by extracellular signaling events and the HIF hypoxia regulators. Note that these data were developed in specific cancer cell lines, where HIF will often be abnormally hyperactive, as a result of accumulated mutations.

DNA flanking each of the inserts was recovered and sequenced, according to the schematic shown in FIG. 10. Intriguingly, many of the inserts were at genetic loci occupied by genes that participate in hypoxia-related responses.

### Example 2

### Identification of Anti-Neoplastic Agents

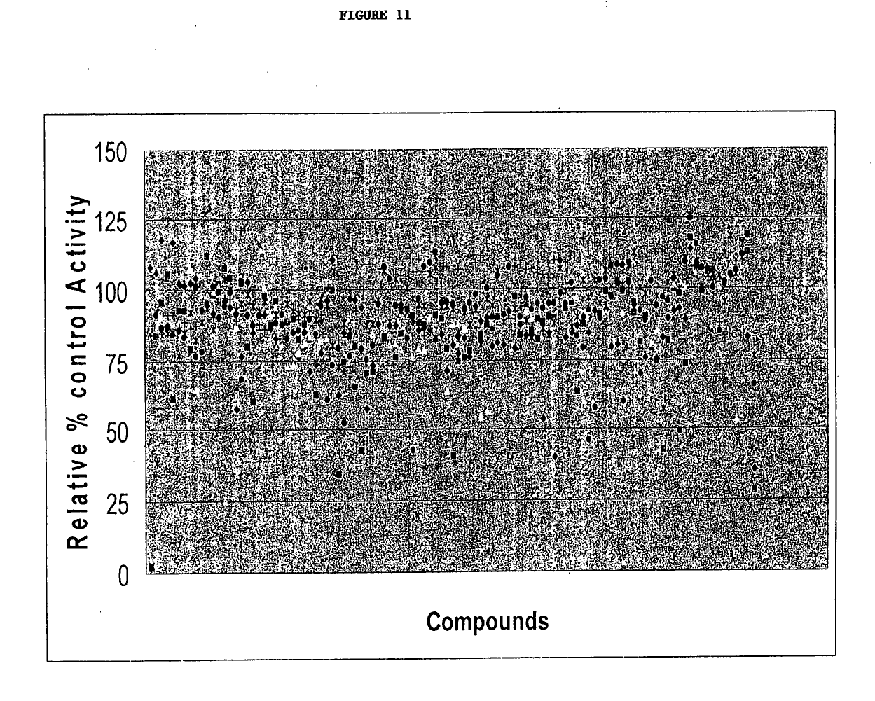

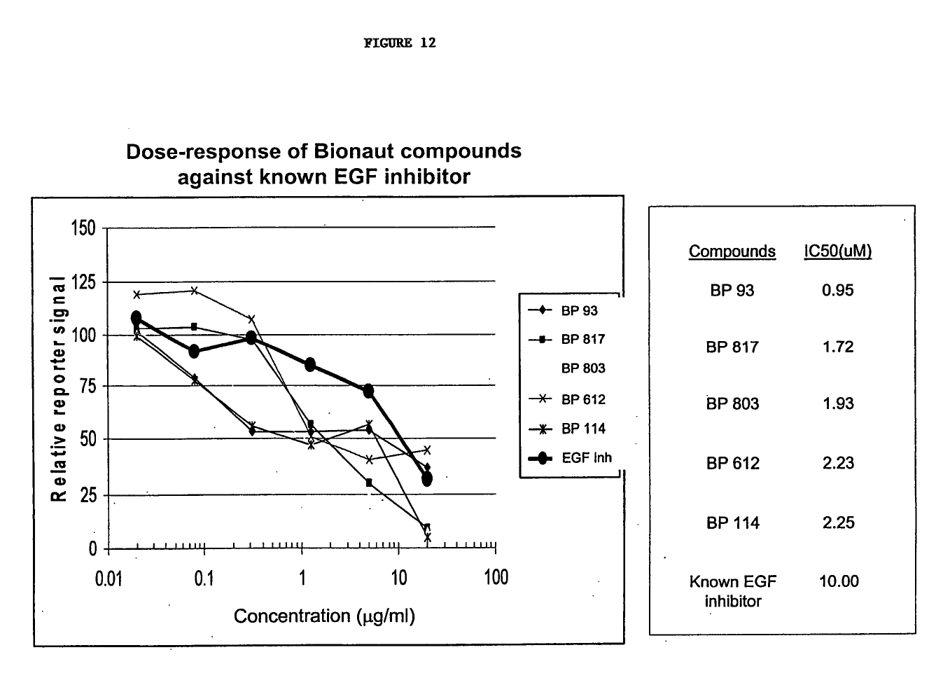

An EGF, HIF responsive cell line was screened for compounds affecting the regulated gene expression. Thousands of compounds were screened. As shown in FIG. 11, most compounds had little or no effect. Those with greatest effect were selected for more detailed analysis. As shown in FIG. 12, many of the most effective compounds have a potency that is equivalent to or greater than that of Iressa, indicating that the cell based assay system provides a rapid system for identifying highly effective anti-neoplastic agents.

As described above, the effective agents may be divided into three broad categories, cardiac glycosides, redox effectors, and steroid signal modulators. Among the cardiac glycosides are ouabain, digitoxigenin, digoxin and lanatoside C. Among the redox effectors are: alexidine dihydrochloride, clofazimine, niclosamide, proadifen hydrochloride, menadione and mefloquine hydrochloride. Among the steroid signal modulators are: bicalutamide, diethylstilbestrol, dienestrol, econazole nitrate, and clotrimazole.

The cell-based assay system was also used to rapidly test combinations of agents. The combination of the redox effector proadifen and the steroid signal modulator lanatoside C was highly effective. Combinations within each class also showed high effectiveness. The combination of mefloquine and clofazimine was highly effective.

### Example 3

### The Identification of New Indications for Known Drugs with HIF-Responsive Reporter Cell Lines

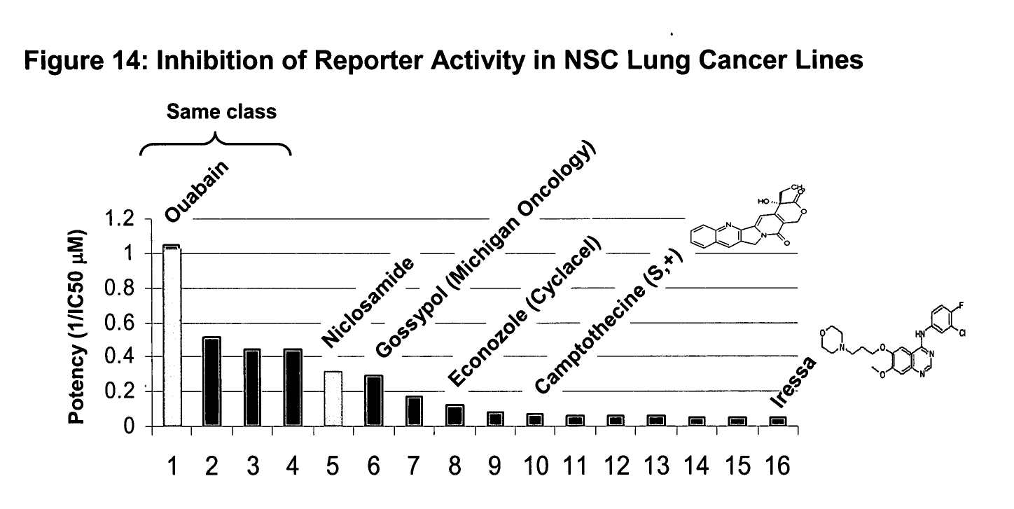

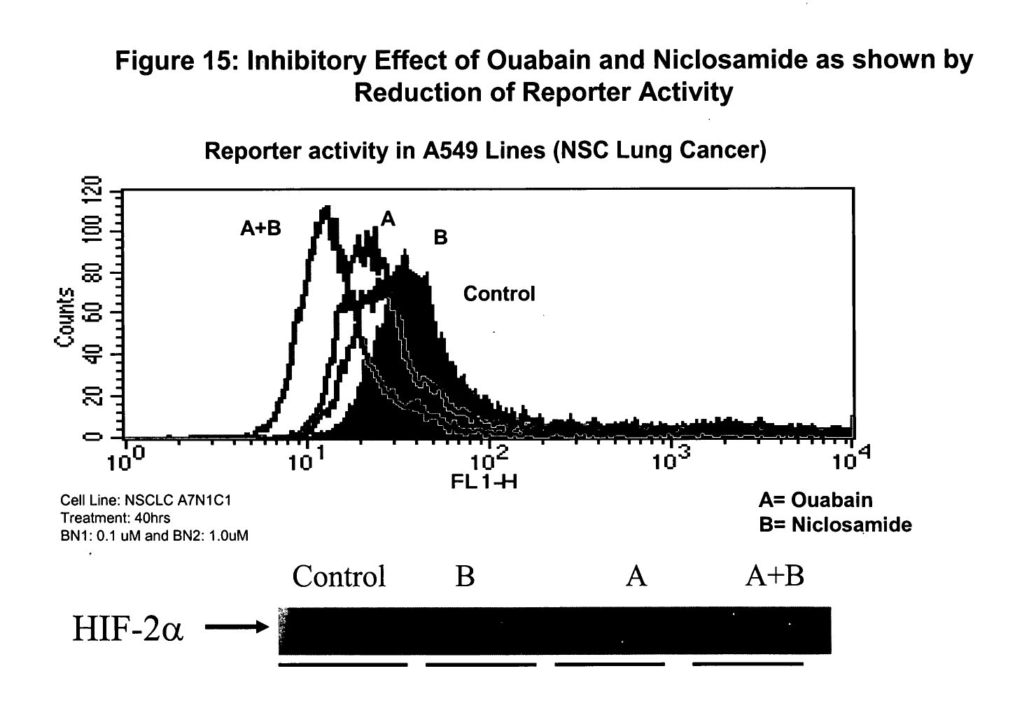

A more detailed analysis of compounds with the greatest effect according to example 2 was performed. The detailed analysis produced two compounds, ouabain and niclosamide, neither of which is currently prescribed in the treatment of cancer as shown in FIG. 14. Surprisingly, the required dose of ouabain for inhibition of reporter activity was well below the dose required for ouabain as a cardiotonic agent. When ouabain and niclosamide were administered in combination to reporter cells, an additive effect was observed as shown in FIG. 15, allowing for even lower dosing of ouabain, while still inhibiting reporter activity. An analogous additive effect was also observed when bicalutamide was administered in combination with either ouabain or niclosamide to inhibit reporter activity.

### Example 4

### The Correlation of Reporter Inhibition of Reporter Activity with Anti-Proliferative Effect

The inhibition of reporter activity was later correlated with an anti-proliferative effect in representative colon, lung, prostate, cervical, renal, uterine, breast, and pancreatic cancer cell lines as shown in table 1. These results demonstrate that the HIF-responsive reporter cell lines generated according to the methods disclosed herein provide a rapid screening assay for identifying novel antiproliferative agents.

TABLE 1

| | Ouabain | Niclosamide |
|---|---|---|
| Cell Line/Type | IC50 (mM) | IC50 (mM) |
| HT-29/Colon | >0.8 | 8.0 |
| HCT-116/Colon | 0.15 | 0.80 |
| A549/Lung | 0.3 | 1.5 |
| SK-MES/Lung | 0.1 | 2.0 |
| LnCap/Prostate | 0.15 | 2.0 |
| DU-145/Prostate | 0.3 | 5.0 |
| PC3/Prostate | 0.15 | 5.0 |
| C-33A/Cervical | 0.10 | 5.0 |
| Caki-1/Renal | 0.15 | 1.5 |
| MES-SA/Uterine | 0.08 | 9.0 |
| MES-SA-DX5/Uterine | 0.08 | 6.0 |
| MCF-7/Breast | 0.10 | 2.5 |
| Panc-1 | 0.15 | 5.0 |

### Example 5

### Combination Therapy Using a Cardiac Glycoside and an Steroidogenesis Inhibitor

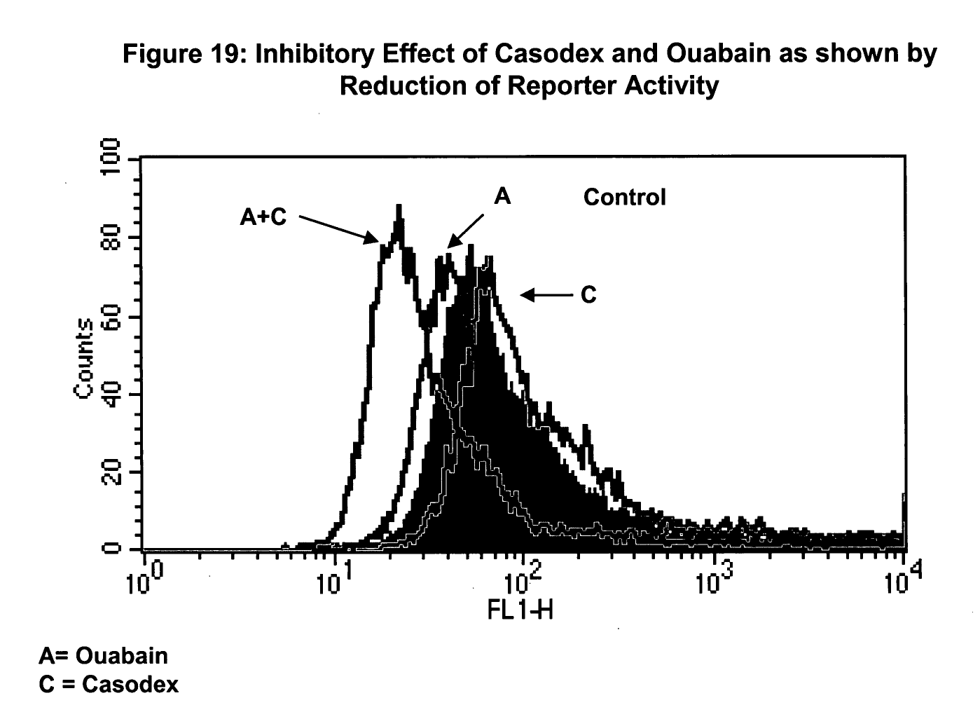

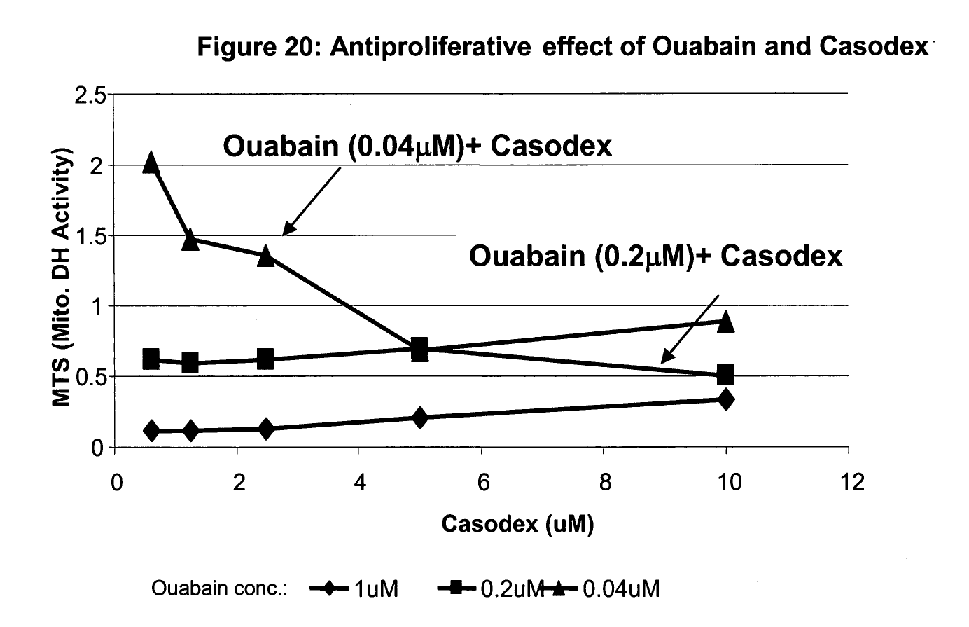

As shown in FIGS. 19 and 20, bicalutamide and ouabain show significant interaction in generating an antiproliferative effect. The ouabain concentration required to achieve an antiproliferative effect in cells of the HT29 colon cancer cell line (FIG. 20) is decreased five-fold in the presence of bicalutamide (Casodex).

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A pharmaceutical formulation comprising:
a. a compound of the niclosamide class of compounds formulated for systemic administration; and
b. a pharmaceutically acceptable excipient.

2. The pharmaceutical formulation of claim 1, wherein the compound of the niclosamide class of compounds is formulated for slow release.

3. The pharmaceutical formulation of claim 1, further comprising a steroid signal modulator.

4. The pharmaceutical composition of claim 1, further comprising a cardiac glycoside.

5. The pharmaceutical composition of claim 4, wherein the cardiac glycoside is ouabain.

6. A method of treating a neoplastic disorder comprising: administering a therapeutically effective amount of a compound of the niclosamide class.

7. The method of claim 6, wherein the neoplastic disorder is a cancer comprising a HIF-positive tumor cell.

8. The method of claim 6, wherein the neoplastic disorder is a cancer selected from among the following: pancreatic cancer, lung cancer, colon cancer, prostate cancer, cervical cancer, renal cancer, uterine cancer and breast cancer.

9. A pharmaceutical formulation comprising:
a. a cardiac glycoside in an amount sufficient to decrease the expression of a HIF-responsive gene and insufficient to achieve an optimal cardiovascular therapeutic effect; and
b. a pharmaceutically acceptable excipient.

10. The pharmaceutical composition of claim 9, wherein the cardiac glycoside is an aglycone form.

11. The pharmaceutical composition of claim 9, wherein the cardiac glycoside is selected from the following: a butenolide form and a pyrone form.

12. The pharmaceutical formulation of claim 9, further comprising a steroid signal modulator.

13. The pharmaceutical formulation of claim 12, wherein the steroid signal modulator is bicalutamide.

14. The pharmaceutical composition of claim 9, further comprising a redox effector.

15. The pharmaceutical composition of claim 14, wherein the redox effector is a member of the niclosamide class of compounds.

16. A method of treating a neoplastic disorder comprising: administering a cardiac glycoside in an amount sufficient to decrease the expression of a HIF-responsive gene and insufficient to achieve an optimal cardiovascular therapeutic effect.

17. The method of claim 16, wherein the neoplastic disorder is a cancer comprising a HIF-positive tumor cell.

18. The method of claim 16, wherein the neoplastic disorder is a cancer selected from among the following: pancreatic cancer, lung cancer, colon cancer, prostate cancer, cervical cancer, renal cancer, uterine cancer and breast cancer.

19. The method of claim 16, wherein the pharmaceutical composition further comprises an additional anti-neoplastic agent.

20. The method of claim 19, wherein the additional anti-neoplastic agent is selected from among: a cardiac glycoside, a redox effector, a steroid signal modulator and an EGF antagonist.

21. The method of claim 20, wherein the redox effector is selected from among: a clofazimine, an electron transport inhibitor, a cytochrome P450 inhibitor and an electron acceptor.

22. The method of claim 20, wherein the redox effector is selected from among: clofazimine, niclosamide, proadifen, mefloquin and menadione.

23. The method of claim 20, wherein the steroid signal modulator is selected from among: an estrogen agonist, a cardiac glycoside and a steroidogenesis inhibitor.

24. The method of claim 20, wherein the steroid signal modulator is selected from among: diethylstilbestrol; dienestrol; digitoxigenin; digoxin; lanatoside C and econazole.

25. The method of claim 20, wherein the steroidogenesis inhibitor is bicalutamide.

26. The method of claim 20, wherein the EGF antagonist is selected from among: gelfitinib and erlotinib.

27. A pharmaceutical composition comprising:
a. a cardiac glycoside;
b. a second active ingredient selected from the group consisting of: a steroid signal modulator, a redox effector and an EGF antagonist; and
c. a pharmaceutically acceptable carrier.

28. A cell comprising: a nucleic acid construct which includes a positive selection marker and a negative selection marker, wherein expression of the positive selection marker and the negative selection marker is regulated by extracellular signaling.
